(19) United States
(12) Patent Application Publication
(10) Pub. No.: US 2011/0039773 A1
(43) Pub. Date: Feb. 17, 2011

(54) BMP Mutants with Decreased Susceptibility to Noggin

(75) Inventors: Moulay Hicham Alaoui-Ismaili, Hopkinton, MA (US); Kening Song, Acton, MA (US)

Correspondence Address: K&L Gates LLP, STATE STREET FINANCIAL CENTER, One Lincoln Street, BOSTON, MA, 02111-2950, US

(73) Assignee: Stryker Biotech, Hopkinton, MA (US)

(21) Appl. No.: 12/746,821

(22) PCT Filed: Dec. 19, 2008
(86) PCT No.: PCT/US08/87720

§ 371 Date: Jun. 8, 2010

Related U.S. Application Data
(60) Provisional application No. 61/008,754, filed on Dec. 21, 2007.

(51) Int. Cl. A61K 38/18; C07K 14/51; C07K 14/475; C07H 21/00; C12N 15/63; A61P 19/08; A61P 19/04

(52) U.S. Cl. 514/8.8; 530/350; 514/7.6; 536/23.5; 435/320.1

(57) ABSTRACT

The present invention provides modified, highly potent bone morphogenetic proteins. In particular, the present invention relates to the observation that BMP-6 and BMP-9 are less susceptible to inhibition by Noggin that are other members of the BMP subfamily of proteins. The present invention features chimeric bone morphogenetic proteins in which the middle portion of BMP-6 or BMP-9 replaces the middle portion of another BMP subfamily protein to cause resistance to inhibition by Noggin or other Noggin-like antagonists. Other embodiments of modified BMPs, compositions and methods of use are also included.

### CROSS-REFERENCE TO RELATED APPLICATIONS

This application claims priority to and the benefit of U.S. Provisional Patent Application No. 61/008,754, filed Dec. 21, 2007, the contents of which are incorporated by reference herein.

### FIELD OF THE INVENTION

The invention relates to the observation that BMP-6 and BMP-9 show greater resistance to inhibition by the protein inhibitor Noggin than do other members of the BMP subfamily. In particular, this invention relates to designed or modified bone morphogenetic proteins with decreased susceptibility to Noggin and Noggin-like proteins, and to methods of making and using compositions utilizing the designed or modified bone morphogenetic proteins.

### BACKGROUND OF THE INVENTION

Bone morphogenetic proteins (BMPs) belong to the superfamily of transforming growth factor β (TGF-β), and control a diverse set of cellular and developmental processes, such as embryonic pattern formation and tissue specification as well as promoting wound healing and repair processes in adult tissues. BMPs were initially isolated by their ability to induce bone and cartilage formation.

BMPs initiate signaling by binding to and bringing together the type I and type II receptor Ser/Thr kinases on the cell surface. This allows the type II receptors to phosphorylate the type I receptor kinases. The type I receptor kinases then phosphorylate members of the Smad family of transcription factors, and the Smads translocate to the nucleus and activate the expression of a number of genes. BMP signaling is inducible upon bone fracture and related tissue injury, leading to bone regeneration and repair. Neighboring cells, on the other hand, selectively secrete BMP antagonists, such as Noggin and Chordin, in response to BMP signaling to allow them to escape from BMP signaling.

Although antagonists may help to provide spatial regulation of the BMP signaling, their action may extend beyond the region where they are secreted and result in reduced BMP activity near or at the bone regeneration site since the antagonists are generally secreted into the extracellular compartment. BMP molecules which have decreased susceptibility to their antagonists would have improved biological activity relative to the native proteins. Such modified BMPs would have therapeutic utility in the field of tissue regeneration, providing potent activity at lower protein levels than the currently used therapeutic doses.

### SUMMARY OF THE INVENTION

It is therefore an object of the present invention to provide designed highly potent bone morphogenetic proteins (BMPs) suitable for therapeutic uses.

It is an object of the present invention to provide designed BMPs with reduced susceptibility to inhibition by their antagonists. Particularly, it is an object of the present invention to provide designed BMPs with reduced susceptibility to inhibition by Noggin and/or Noggin-like proteins.

It is a further object of the invention to provide nucleic acid sequences which encode the designed BMPs of the invention and methods of using such nucleic acid sequences for producing the designed BMPs of the invention.

Thus, in one aspect, the present invention features a protein comprising a chimera of a TGF-beta superfamily protein and wild-type BMP-6, wherein one or more amino acid sequences of wild-type BMP-6 replace amino acid sequences at corresponding residue positions of the TGF-beta superfamily protein and wherein the chimera is resistant to inhibition by an antagonist of the TGF-beta superfamily protein and exhibits greater biological activity than that of the TGF-beta superfamily protein, and further wherein the TGF-beta superfamily protein is not wild-type BMP-6. In one embodiment, the antagonist is selected from the group of cysteine knot protein antagonists consisting of: Noggin, Noggin-like proteins, Cerberus/DAN family proteins, Chordin/SOG family proteins and functional equivalents of any of the foregoing.

In another aspect, the present invention features a protein comprising a chimera of a TGF-beta superfamily protein and wild-type BMP-9, wherein one or more amino acid sequences of wild-type BMP-9 replace amino acid sequences at corresponding residue positions of the TGF-beta superfamily protein and wherein the chimera is resistant to inhibition by an antagonist of the TGF-beta superfamily protein and exhibits greater biological activity than that of the TGF-beta superfamily protein, and further wherein the TGF-beta superfamily protein is not wild-type BMP-9. In one embodiment, the antagonist is selected from the group of cysteine knot protein antagonists consisting of: Noggin, Noggin-like proteins, Cerberus/DAN family proteins, Chordin/SOG family proteins and functional equivalents of any of the foregoing.

In another aspect, the present invention features a protein comprising a modified bone morphogenetic protein (BMP) or a modified growth differentiation factor (GDF), wherein the modified BMP or GDF is less inhibited by an antagonist than a corresponding unmodified wild-type BMP or wild-type GDF and has greater biological activity in the presence of the antagonist than the corresponding unmodified wild-type BMP or GDF. In one embodiment, the antagonist is selected from the group of cysteine knot protein antagonists consisting of: Noggin, Noggin-like proteins, Cerberus/DAN family proteins, Chordin/SOG family proteins and functional equivalents of any of the foregoing.

In another aspect, the present invention relates to a protein resistant to inhibition by Noggin or a Noggin-like protein, said protein comprising a modified bone morphogenetic protein (BMP) or a modified growth differentiation factor (GDF), wherein at least three replacement amino acids selected from the group consisting of Val45, Gln48, Asp49, Lys50, Gln53, Ile57, Lys60, Gly61, Ala63, Asn65, Tyr66, Asp68, Glu70, Ser72, Asn76, Ala77, His 78, Met79 and Asn80 of human BMP-6 replace at least three amino acids in corresponding positions of a wild-type BMP or a wild-type GDF, and also wherein said at least three replacement amino acids differ from said at least three amino acids in corresponding positions of said wild-type BMP or said wild-type GDF.

In another aspect, the present invention relates to a protein resistant to inhibition by Noggin or a Noggin-like protein, said protein comprising a modified bone morphogenetic protein (BMP) or a modified growth differentiation factor (GDF), wherein Val45 to Asn80 (Residues 45-80 of SEQ ID NO:3) of wild-type human BMP-6 replaces a corresponding segment of an otherwise wild-type human BMP or human GDF and the corresponding segment that has been replaced is not identical to said Val45 to Asn80 of wild-type BMP-6.

In another aspect, the present invention relates to a protein resistant to inhibition by Noggin or a Noggin-like protein, said protein comprising a modified bone morphogenetic protein (BMP) or a modified growth differentiation factor (GDF), wherein Val76 to Thr111 (Residues 76-111 of SEQ ID NO:46) of wild-type human BMP-9 replaces a corresponding segment of an otherwise wild-type human BMP or human GDF and the corresponding segment that has been replaced is not identical to said Val76 to Thr111 (Residues 76-111 of SEQ ID NO:46) of wild-type BMP-9.

In yet another aspect, the present invention relates to a modified BMP-2 comprising at least one amino acid substitution selected from the group consisting of D22S, S24Q, V26L, N29Q, V33I, P36K, H39A, F41N, H44D, P48S, A52N, D53A, and L55M, wherein all other residues are identical to wild-type BMP-2 or share at least 93% amino acid sequence identity with the conserved cysteine domain of the C-terminal region of wild-type BMP-2.

In yet another aspect, the present invention relates to a modified BMP-2 comprising an amino acid substitution at least one of the following positions: D22, S24, V26, N29, V33, P36, H39, F41, H44, P48, A52, D53, and L55, wherein the modified BMP-2 shares at least 93% amino acid sequence identity with the conserved cysteine domain of the C-terminal region of wild-type BMP-2.

In another aspect, the present invention relates to a modified BMP-4 comprising at least one amino acid substitution selected from the group consisting of D24S, S26Q V28L, N31Q, V35I, P38K, Q41A, F43N, H46D, D48E, P50S, A54N, D55A, and L57M, wherein all other residues are identical to wild-type BMP-4 or share at least 93% amino acid sequence identity with the conserved cysteine domain of the C-terminal region of wild-type BMP-4.

In another aspect, the present invention relates to a modified BMP-4 comprising an amino acid substitution at least one of the following positions: D24, S26, V28, N31, V35, P38, Q41, F43, H46, D48, P50, A54, D55, and L57, wherein the modified BMP-4 shares at least 93% amino acid sequence identity with the conserved cysteine domain of the C-terminal region of wild-type BMP-4.

In another aspect, the present invention relates to a modified BMP-5 comprising at least one amino acid substitution selected from the group consisting of R41Q, E53K, and F58N, wherein all other residues are identical to wild-type BMP-5 or share at least 93% amino acid sequence identity with the conserved cysteine domain of the C-terminal region of wild-type BMP-5.

In another aspect, the present invention relates to a modified BMP-5 comprising an amino acid substitution at least one of the following positions: R41, E53, and F58, wherein the modified BMP-5 shares at least 93% amino acid sequence identity with the conserved cysteine domain of the C-terminal region of wild-type BMP-5.

In another aspect, the present invention relates to a modified BMP-7 comprising at least one amino acid substitution selected from the group consisting of R48Q, E60K, E68D, A72S and S77A, wherein all other residues are identical to wild-type BMP-7 or share at least 89% amino acid sequence identity with the conserved cysteine domain of the C-terminal region of wild-type BMP-7.

In another aspect, the present invention relates to a modified BMP-7 comprising an amino acid substitution at least one of positions: R48, E60, E68, A72 and S77, wherein the modified BMP-7 shares at least 89% amino acid sequence identity with the conserved cysteine domain of the C-terminal region of wild-type BMP-7.

In another aspect, the present invention relates to a modified BMP-7 comprising at least two amino acid substitutions selected from the group consisting of R48Q, E60K, Y65N, E68D, A72S and S77A, wherein all other residues are identical to wild-type BMP-7 or share at least 89% amino acid sequence identity with the conserved cysteine domain of the C-terminal region of wild-type BMP-7.

In another aspect, the present invention relates to a modified BMP-7 comprising an amino acid substitution at least two of positions: R48, E60, Y65, E68, A72 and S77, wherein the modified BMP-7 shares at least 89% amino acid sequence identity with the conserved cysteine domain of the C-terminal region of wild-type BMP-7.

In another aspect, the present invention relates to a modified BMP-7 comprising at least three amino acid substitutions selected from the group consisting of R48Q, E60K, Y65N, E68D, A72S, S77A, and Y78H, wherein all other residues are identical to wild-type BMP-7 or share at least 89% amino acid sequence identity with the conserved cysteine domain of C-terminal region of wild-type BMP-7.

In another aspect, the present invention relates to a modified BMP-7 comprising an amino acid substitution at least three of positions: R48, E60, Y65, E68, A72, S77, and Y78 wherein the modified BMP-7 shares at least 89% amino acid sequence identity with the conserved cysteine domain of the C-terminal region of wild-type BMP-7.

In another aspect, the present invention relates to a modified GDF-5 comprising at least one amino acid substitution selected from the group consisting of N27S, K29Q, M31L, D34Q, L41K, E42G, E44A, F46N, H47Y, E49D, L51E, E53S, R57N, S58A, L60M, and E61N, wherein all other residues are identical to wild-type GDF-5 or share at least 87% amino acid sequence identity with the conserved cysteine domain of the C-terminal region of wild-type GDF-5.

In another aspect, the present invention relates to a modified GDF-5 comprising an amino acid substitution at least one of the following positions: N27, K29, M31, D34, L41, E42, E44, F46, H47, E49, L51, E53, R57, S58, L60, and E61, wherein all other residues are identical to wild-type GDF-5 or share at least 87% amino acid sequence identity with the conserved cysteine domain of the C-terminal region of wild-type GDF-5.

In another aspect, the present invention relates to a modified GDF-6 comprising at least one amino acid substitution selected from the group consisting of N27S, K29Q, E30D, D34Q, L41K, E42G, E44A, Y46N, H47Y, E48D, V51E, D53S, R57N, S58A, L60M, and E61N, wherein all other residues are identical to wild-type GDF-6 or share at least 97% amino acid sequence identity with the conserved cysteine domain of the C-terminal region of wild-type GDF-6.

In another aspect, the present invention relates to a modified GDF-6 comprising an amino acid substitution at least one of the following positions: N27, K29, E30, D34, L41, E42, E44, Y46, H47, E48, V51, D53, R57, S58, L60, and E61, wherein all other residues are identical to wild-type GDF-6 or share at least 97% amino acid sequence identity with the conserved cysteine domain of the C-terminal region of wild-type GDF-6.

In another aspect, the present invention relates to a modified GDF-7 comprising at least one amino acid substitution selected from the group consisting of D36S, K38Q, E39D, D43Q, L50K, D51G, E53A, Y55N, H56Y, E58D, L60E, D62S, R66N, S67A, L69M, and/or E70N wherein all other residues are identical to wild-type GDF-7 or share at least 87% amino acid sequence identity with the conserved cysteine domain of the C-terminal region of wild-type GDF-7.

In another aspect, the present invention relates to a modified GDF-7 comprising an amino acid substitution at least one of the following positions: D36S, K38, E39, D43, L50, D51, E53, Y55, H56, E58, L60, D62, R66, S67, L69, and/or E70 wherein all other residues are identical to wild-type GDF-7 or share at least 87% amino acid sequence identity with the conserved cysteine domain of the C-terminal region of wild-type GDF-7.

In yet another aspect, the present invention relates to a method for modulating inhibition of a modified bone morphogenetic protein (BMP) or a modified growth differentiation factor (GDF) by Noggin or a Noggin-like protein, the method comprising the step of providing a modified bone morphogenetic protein (BMP) or a modified growth differentiation factor (GDF), wherein at least two replacement amino acids selected from the group consisting of Val45, Gln48, Asp49, Lys50, Gln53, Ile57, Lys60, Gly61, Ala63, Asn65, Tyr66, Asp68, Glu70, Ser72, Asn76, Ala77, Met79 and Asn80 of human BMP-6 replace at least two amino acids in corresponding positions of a wild-type BMP or a wild-type GDF, and said at least two replacement amino acids differ from said at least two amino acids in corresponding positions of said wild-type BMP or said wild-type GDF, and also wherein the providing step results in a modulation of inhibition of said BMP or GDF by Noggin or a Noggin-like protein as compared to the BMP or GDF wild-type counterpart.

In another aspect, the present invention relates to a method for modulating the inhibition of a bone morphogenetic protein (BMP) or a growth differentiation factor (GDF) by Noggin or a Noggin-like protein, the method comprising the step of providing a modified BMP or GDF protein, said modified protein resulting from replacing a corresponding segment of a human BMP or a human GDF with Val45 to Asn80 (Residues 45-80 of SEQ ID NO:3) of wild-type human BMP-6, wherein the corresponding segment that has been replaced is not identical to said Val45 to Asn80 of wild-type BMP-6, and wherein the providing step results in a modulation of inhibition of said BMP or GDF by Noggin or a Noggin-like protein as compared to the BMP or GDF wild-type counterpart.

In another aspect, the present invention relates to a non-naturally occurring peptide comprising an N-terminal and a C-terminal amino acid sequence corresponding to a wild-type BMP or a GDF protein, wherein each of the N-terminal and the C-terminal amino acid sequences of said non-naturally occurring peptide shares at least 97% amino acid sequence identity with the wild-type BMP or GDF protein and further wherein said non-naturally occurring peptide has at least two amino acid residues at positions corresponding to BMP-6 amino acid residues selected from the group consisting of Val45, Ser46, Gln48, Asp49, Lys50, Gln53, Ile57, Lys60, Gly61, Ala63, Asn65, Tyr66, Asp68, Glu70, Ser72, Asn76, Ala77, Met79 and Asn80, with the proviso that said BMP is not BMP-6. In one embodiment, the N-terminal or C-terminal amino acid sequence of the non-naturally occurring peptide is identical to the wild-type BMP or GDF protein. In another embodiment, the non-naturally occurring peptide displays reduced inhibition of bioactivity by a Noggin or Noggin-like protein as compared to its wild-type BMP or GDF counterpart. In yet another embodiment, the present invention provides for a pharmaceutical composition comprising the non-naturally occurring peptide admixed with a pharmaceutically-acceptable carrier.

In yet another aspect, the present invention relates to a method for modulating inhibition of a modified bone morphogenetic protein (BMP) or a modified growth differentiation factor (GDF) by Noggin or a Noggin-like protein, the method comprising the step of providing a modified bone morphogenetic protein (BMP) or a modified growth differentiation factor (GDF), wherein at least two replacement amino acids selected from the group consisting of Asn77, Glu79, Ile81, Asp84, Ser85, Ile88, Lys91, Glu92, Glu94, Tyr96, Glu97, Lys99, Gly101, Phe103, Ala107, Asp108, Asp109, Val110, or Thr111 of human BMP-9 replace at least two amino acids in corresponding positions of a wild-type BMP or a wild-type GDF, and said at least two replacement amino acids differ from said at least two amino acids in corresponding positions of said wild-type BMP or said wild-type GDF, and also wherein the providing step results in a modulation of inhibition of said BMP or GDF by Noggin or a Noggin-like protein as compared to the BMP or GDF wild-type counterpart.

In another aspect, the present invention relates to a method for modulating the inhibition of a bone morphogenetic protein (BMP) or a growth differentiation factor (GDF) by Noggin or a Noggin-like protein, the method comprising the step of providing a modified BMP or GDF protein, said modified protein resulting from replacing a corresponding segment of a human BMP or a human GDF with Val76 to Thr111 of wild-type human BMP-9, wherein the corresponding segment that has been replaced is not identical to said Val76 to Thr111 of wild-type BMP-9, and wherein the providing step results in a modulation of inhibition of said BMP or GDF by Noggin or a Noggin-like protein as compared to the BMP or GDF wild-type counterpart.

In another aspect, the present invention relates to a non-naturally occurring peptide comprising an N-terminal and a C-terminal amino acid sequence corresponding to a wild-type BMP or a GDF protein, wherein each of the N-terminal and the C-terminal amino acid sequences of said non-naturally occurring peptide shares at least 97% amino acid sequence identity with the wild-type BMP or GDF protein and further wherein said non-naturally occurring peptide has at least two amino acid residues at positions corresponding to BMP-9 amino acid residues selected from the group consisting of Asn77, Glu79, Ile81, Asp84, Ser85, Ile88, Lys91, Glu92, Glu94, Tyr96, Glu97, Lys99, Gly101, Phe103, Ala107, Asp108, Asp109, Val110, or Thr111 with the proviso that said BMP is not BMP-9. In one embodiment, the N-terminal or C-terminal amino acid sequence of the non-naturally occurring peptide is identical to the wild-type BMP or GDF protein. In another embodiment, the non-naturally occurring peptide displays reduced inhibition of bioactivity by a Noggin or Noggin-like protein as compared to its wild-type BMP or GDF counterpart.

In yet another embodiment, the present invention provides for a pharmaceutical composition comprising the non-naturally occurring peptide admixed with a pharmaceutically-acceptable carrier.

In yet another embodiment, the present invention provides for nucleic acids encoding modified BMP or GDF proteins of the invention.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1 shows the amino acid sequence of a mature human BMP-7 protein (SEQ ID NO:).

FIG. 2 shows the amino acid sequence of a mature human BMP-2 protein (SEQ ID NO:2).

FIG. 3 shows the amino acid sequence of a mature human BMP-6 protein (SEQ ID NO:3).

FIG. 4 shows the amino acid sequence of a mature human BMP-4 protein (SEQ ID NO:4).

FIG. 5 shows the amino acid sequence of a mature human BMP-5 protein (SEQ ID NO:5).

FIG. 6 shows the amino acid sequence of a mature human GDF-5 protein (SEQ ID NO:6).

FIG. 7 shows the amino acid sequence of a mature human GDF-6 protein (SEQ ID NO:7).

FIG. 8 shows the amino acid sequence of a mature human GDF-7 protein (SEQ ID NO:8).

FIG. 9 is a schematic depiction of mechanisms of BMP and TGF-β signaling. As shown therein, upon binding of the ligands, specific pairs of the two types of Ser/Thr kinase receptors heterodimerize, one type I and the other type II. Type II kinase then phosphorylates the type I receptor kinase upon heterodimerization to allow type I receptor kinase to bind ATP and Smad protein substrates for signaling. The ligands can be divided into three groups according to signaling pathways (Avidin, TGF-β, and BMPs) or two groups according to sequence similarity and the mode of binding (TGF-β/Avidin, and BMPs).

FIG. 10 depicts a BMP-7-Noggin structural complex. As shown therein, the noggin binding site on BMP-7 spans over both the type-I and type-II receptor binding sites. The Noggin complex is depicted in black (right) and gray striped (left) and the BMP-7 complex is depicted in white (right) and dotted (left).

FIG. 11 is a comparative analysis of the susceptibility of BMPs to Noggin in a ROS Alkaline Phosphatase Assay and depicts percentage inhibition of several exemplary BMP family members by Noggin. BMP-6 is the least susceptible to inhibition by Noggin.

FIG. 12A is a comparative analysis of the susceptibility of BMPs to Noggin in a luciferase reporter gene assay and depicts percentage inhibition of several exemplary BMP and GDF family members by Noggin, measured by BMP-induced luciferase inhibition in A-549-BRE cells. BMP-6 is the least susceptible to inhibition by Noggin, followed by BMP-7, then BMP-2, with BMP-4 being the most susceptible to inhibition by Noggin.

FIG. 12B is a comparative analysis of the susceptibility of BMPs to Noggin in a QPCR-based ID-1 gene expression assay and depicts the relative quantity (RQ) of ID-1 mRNA following treatment of human bone marrow derived mesenchymal stem cells (hMSCs) with several exemplary BMP family members in the presence or absence of Noggin. BMP-6 is the least susceptible to inhibition by Noggin, followed by BMP-7, then BMP-2, with BMP-4 being the most susceptible to inhibition by Noggin.

FIG. 13 shows the BMP-6/BMP-7 chimeras that were made to determine which regions of BMP-6 are important for reduced susceptibility to Noggin. In the first chimera, amino acids 1-40 of BMP-7 (residues 1-40 of SEQ ID NO:1) were replaced with the corresponding amino acids of BMP-6. In the second chimera, amino acids 45-80 of BMP-7 (residues 45-80 of SEQ ID NO:1) were replaced with the corresponding amino acids of BMP-6. In the third chimera, amino acids 90-120 of BMP-7 (residues 90-120 of SEQ ID NO:1) were replaced with corresponding amino acids of BMP-6.

FIG. 14 shows the extent of inhibition by different concentrations of Noggin (N1 low, N2 medium, and N3 high) on wild-type BMP-6 and wild-type BMP-7 and the three BMP-6/BMP-7 chimeras. “40-1” indicates the chimera in which amino acids 1-40 of BMP-6 (residues 1-40 of SEQ ID NO:3) replaced the corresponding amino acids of BMP-7. “80-1” indicates the chimera in which amino acids 45-80 of BMP-6 (residues 45-80 of SEQ ID NO:3) replaced the corresponding amino acids of BMP-7. “90-1” indicates the chimera in which amino acids 90-120 of BMP-6 (residues 90-120 of SEQ ID NO:3) replaced the corresponding amino acids of BMP-7. Among the chimeras tested, the chimera “80-1” is the most resistant to Noggin inhibition.

FIG. 15 is an alignment of protein sequences of mature BMP-6, GDF-5, GDF-6 and GDF-7.

FIG. 16 is an alignment of protein sequences of mature BMP-6, BMP-7 and BMP-5.

FIG. 17 is an alignment of protein sequences of mature BMP-6, BMP-2 and BMP-4.

FIG. 18 depicts the locations of preferred amino acid substitutions in mature BMP-7. The presence of all substitutions is depicted in SEQ ID NO:9.

FIG. 19 depicts the locations of preferred amino acid substitutions in mature BMP-5. The presence of all substitutions is depicted in SEQ ID NO:10.

FIG. 20A depicts the locations of amino acid differences between BMP-6 and BMP-2, which are also preferred positions for amino acid substitutions in mature BMP-2. If all substitutions are made to BMP-2, the resulting sequence is depicted in SEQ ID NO:11.

FIG. 20B depicts those amino acid differences depicted in FIG. 20A, for which BMP-7 also differs from BMP-6. These are the most preferred positions for substitutions in BMP-2. If all preferred substitutions are made, the resulting sequence is depicted in SEQ ID NO:12.

FIG. 21A depicts the locations of amino acid differences between BMP-6 and BMP-4, which are also preferred positions for amino acid substitutions in mature BMP-4. If all preferred substitutions are made, the resulting sequence is depicted in SEQ ID NO:13.

FIG. 21B depicts those amino acid differences depicted in FIG. 21A, for which BMP-7 also differs from BMP-6. These are the most preferred positions for amino acid substitutions in mature BMP-4. If all preferred substitutions are made, the resulting sequence is depicted in SEQ ID NO:14.

FIG. 22A depicts the locations of amino acid differences between BMP-6 and GDF-5, which are also preferred positions for amino acid substitutions in mature GDF-5. If all preferred substitutions are made, the resulting sequence is depicted in SEQ ID NO:15.

FIG. 22B depicts those amino acid differences depicted in FIG. 22A, for which BMP-7 also differs from BMP-6. These are the most preferred positions for amino acid substitutions in mature GDF-5. If all preferred substitutions are made, the resulting sequence is depicted in SEQ ID NO:16.

FIG. 23A depicts the locations of amino acid differences between BMP-6 and GDF-6, which are also preferred positions for amino acid substitutions in mature GDF-6. If all preferred substitutions are made, the resulting sequence is depicted in SEQ ID NO:17.

FIG. 23B depicts those amino acid differences depicted in FIG. 23A, for which BMP-7 also differs from BMP-6. These are the most preferred positions for amino acid substitutions in mature GDF-6. If all the preferred substitutions are made, the resulting sequence is depicted in SEQ ID NO:18.

FIG. 24A depicts the locations of amino acid differences between BMP-6 and GDF-7, which are also preferred positions for amino acid substitutions in mature GDF-7. If all preferred substitutions are made, the resulting sequence is depicted in SEQ ID NO:19.

FIG. 24B depicts those amino acid differences depicted in FIG. 24A, for which BMP-7 also differs from BMP-6. These are the most preferred positions for amino acid substitutions in mature GDF-7. If all the preferred substitutions are made, the resulting sequence is depicted in SEQ ID NO:20.

FIG. 25 depicts the dose-dependent alkaline phosphatase activity of ROS 17/2.8 cells following treatment with various growth factors.

FIG. 26 depicts the EC50 values for various growth factors in inducing alkaline phosphatase activity in ROS 17/2.8 cells. The values were derived from non-linear regression of the mean optical density of samples. IC50 values of noggin for inhibiting the activity of various growth factors is also presented.

FIGS. 27A-F are bar graphs depicting the levels of expression of transcripts of the Id-1, dlx-5, Sp-7, msx-2, noggin, and alkaline phosphatase gene as determined through quantitative PCR in response to treatment of hMSCs with either BMP-6 or BMP-7 proteins, in the presence or absence of Noggin. BMP-6 is more resistant to Noggin inhibition than BMP-7.

FIG. 28 is a line graph depicting the percent inhibition of purified BMP-6, BMP-7, and the BMP-7 variant “80-1” as function of the concentration of noggin. The chimera “80-1” is more resistant to Noggin than wild-type BMP-7. The resistance to Noggin for wild-type BMP-6 and the chimera “80-1” is comparable.

FIG. 29A depicts those amino acid residues between positions 48-78 of wild-type BMP-7 (residues 48-48 of SEQ ID NO:1) and the BMP-7 “80-1” mutant (SEQ ID NO:32) that differ between these two proteins. Also depicted are point mutations made in the BMP-7 “80-1” mutant to revert particular residues to that found in wild-type BMP-7 in order to create the BMP-7 revertant mutants REVQ48R (SEQ ID NO:33), REV K60E (SEQ ID NO:34), REV N65Y (SEQ ID NO:35), REV D68E (SEQ ID NO:36), REVS72A (SEQ ID NO:37), REVA77S (SEQ ID NO:38), and REVH78Y (SEQ ID NO:39). Also shown are these mutants' relative activity in the presence of noggin at three concentrations: 100 ng/ml, 500 ng/ml, and 1000 mg/ml.

FIG. 29B shows in bar graph form the relative activity of BMP-6, BMP-7, “80-1,” and the revertant mutants enumerated in FIG. 29A in the presence of noggin at three concentrations: 100 ng/ml, 500 ng/ml, and 1000 mg/ml as previously represented in FIG. 29A.

FIG. 30A depicts those amino acid residues between positions 48-78 of BMP-7 (residues 48-78 of SEQ ID NO:1) and their corresponding residues in BMP-6 and depicts point mutations made in the BMP-7 sequence to make BMP-7 mutants BMP-7 E60K (SEQ ID NO:21), BMP-7 Y65N (SEQ ID NO:30), BMP-7 Y78H (SEQ ID NO:32), and BMP-7 R48Q/E60K/Y65N (SEQ ID NO:25). Also shown are these mutants' relative activity in the presence of noggin at three concentrations: 100 ng/ml, 500 ng/ml, and 1000 mg/ml.

FIG. 30B shows in bar graph form the relative activity of BMP-6, BMP-7, and the BMP-7 mutants enumerated in FIG. 30A in the presence of noggin at three concentrations: 100 ng/ml, 500 ng/ml, and 1000 mg/ml as previously represented in FIG. 30A.

FIG. 31A is an alignment of the portion of each of human BMP-2, BMP-4, BMP-5, BMP-7, and BMP-9 that corresponds with residues 49-73 of human BMP-6.

FIG. 31B depicts in bar graph form the relative activity of BMP-2, BMP-6, BMP-7, and the purified BMP-7 E60K mutant in the presence of three concentrations of noggin: 100 ng/ml, 500 ng/ml, and 1000 ng/ml.

FIG. 31C depicts in bar graph form the relative activity of BMP-6, BMP-2, and the BMP-2 P36K mutant in the presence of three concentrations of noggin: 100 ng/ml, 500 ng/ml, and 1000 ng/ml.

FIG. 32 is a chart showing amino acid residues differing between positions 48-78 of BMP-6 and BMP-7 and depicts point mutations required to make the BMP-7 mutants BMP-7 R48Q/S77A (SEQ ID NO:22), BMP-7 R48Q/E60K (SEQ ID NO:23), BMP-7 R48Q/E60K/S77A (SEQ ID NO:24), BMP-7 R48Q/E60K/Y65N (SEQ ID NO:25), BMP-7 E60K/Y65N (SEQ ID NO:26), BMP-7 E60K/Y65N/A72S (SEQ ID NO:27), BMP-7 R48K/E60K/Y65N/A72S (SEQ ID NO:28) and BMP-7 Y65N/Y78H (SEQ ID NO:29).

FIG. 33 is a table depicting a subset of the various BMP-7 mutants created, their level of expression in HEK-293T cells, their level of activity in a luciferase reporter gene assay (as described herein) and their level of inhibition in the presence of noggin.

FIG. 34 shows in bar graph format the percent inhibition of various BMP-7 mutants in the presence of three concentrations of noggin: 100 ng/ml, 500 ng/ml, and 1000 ng/ml.

FIG. 35 shows the amino acid sequence for the mature BMP-9 protein.

### DETAILED DESCRIPTION OF THE INVENTION

The present invention provides designed or modified BMPs with improved properties relating to BMP signaling. The invention relates to the observation that BMP-6 and BMP-9 each show greater resistance to inhibition by the protein inhibitor Noggin than do other members of the BMP and GDF family. In particular, this invention relates to designed or modified bone morphogenetic proteins with decreased susceptibility to Noggin and Noggin-like proteins, and to methods of making and using compositions utilizing the designed or modified bone morphogenetic proteins. In yet another aspect, the present invention provides therapeutic compositions comprising the modified BMPs of the invention. Thus, the present invention represents a significant advance in BMP therapeutics.

Various aspects of the invention are described in further detail in the following subsections. The use of subsections is not meant to limit the invention. Each subsection may apply to any aspect of the invention.

### Bone Morphogenetic Proteins

BMPs belong to the TGF-β superfamily. TGF-β superfamily proteins are cytokines characterized by six-conserved cysteine residues (Lander et al., (2001) Nature, 409:860-921). The human genome contains about 42 open reading frames encoding TGF-β superfamily proteins. TGF-β superfamily proteins can at least be divided into the BMP subfamily and the TGF-β subfamily based on sequence similarity and the specific signaling pathways that they activate. The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1), and GDF-8 (myostatin). BMPs are sometimes referred to as Osteogenic Protein (OPs), Growth Differentiation Factors (GDFs), or Cartilage-Derived Morphogenetic Proteins (CDMPs). BMPs are also present in other animal species. Furthermore, there is some allelic variation in BMP sequences among different members of the human population. As used herein, “BMP subfamily,” “BMPs,” “BMP ligands” and grammatical equivalents thereof refer to the BMP subfamily members, unless specifically indicated otherwise.

The TGF-β subfamily includes, but is not limited to, TGFs (e.g., TGF-β1, TGF-β2, and TGF-β3), activins (e.g., activin A) and inhibins, macrophage inhibitory cytokine-1 (MIC-1), Mullerian inhibiting substance, anti-Mullerian hormone, and glial cell line derived neurotrophic factor (GDNF). As used herein, “TGF-β subfamily,” “TGF-βs,” “TGF-β ligands” and grammatical equivalents thereof refer to the TGF-β subfamily members, unless specifically indicated otherwise.

The TGF-β superfamily is in turn a subset of the cysteine knot cytokine superfamily. Additional members of the cysteine knot cytokine superfamily include, but are not limited to, platelet derived growth factor (PDGF), vascular endothelial growth factor (VEGF), placenta growth factor (P1GF), Noggin, neurotrophins (BDNF, NT3, NT4, and βNGF), gonadotropin, follitropin, lutropin, interleukin-17, and coagulogen.

Structurally, BMPs are dimeric cysteine knot proteins. Each BMP monomer comprises multiple intramolecular disulfide bonds. An additional intermolecular disulfide bond mediates dimerization in most BMPs. BMPs may form homodimers. Some BMPs may form heterodimers.

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et al. (2001) Growth Factors, 18:251-259).

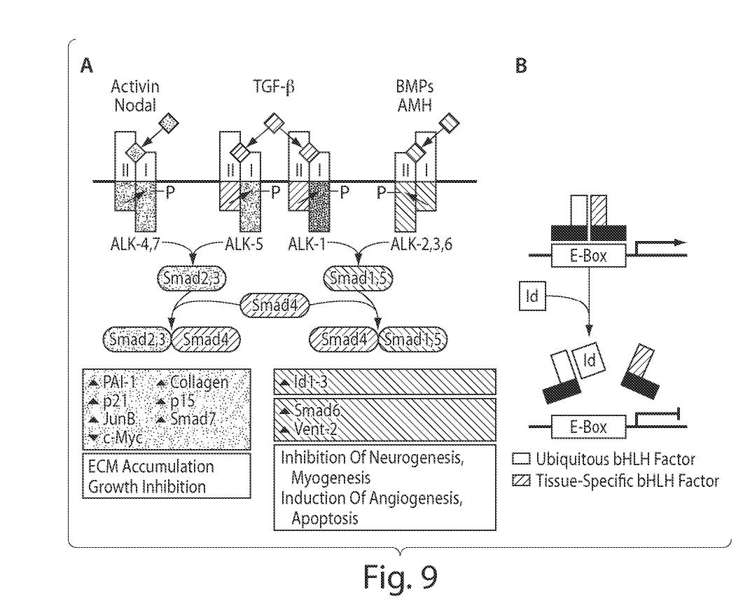

BMP signal transduction is initiated when a BMP dimer binds two type I and two type II serine/threonine kinase receptors. Type I receptors include, but are not limited to, ALK-1, ALK-2 (also called ActRla or ActRI), ALK-3 (also called BMPRIa), and ALK-6 (also called BMPRIb). Type II receptors include, but are not limited to, ActRIIa (also called ActRII), ActRIIb, and BMPRII. The human genome contains 12 members of the receptor serine/threonine kinase family, including 7 type I and 5 type II receptors, all of which are involved in TGF-β signaling (Manning et al., (2002) Science, 298:1912-1934, the disclosures of which are hereby incorporated by reference). Following BMP binding, the type II receptors phosphorylate the type I receptors, the type I receptors phosphorylate members of the Smad family of transcription factors, and the Smads translocate to the nucleus and activate the expression of a number of genes. Mechanisms of BMP and TGF-β signaling are further illustrated in FIG. 9.

BMPs also interact with inhibitors, soluble receptors, and decoy receptors, including, but not limited to, BAMBI (BMP and activin membrane bound inhibitor), BMPER (BMP-binding endothelial cell precursor-derived regulator), Cerberus, cordin, cordin-like, Dan, Dante, follistatin, follistatin-related protein (FSRP), ectodin, gremlin, Noggin, protein related to Dan and cerberus (PRDC), sclerostin, sclerostin-like, SOG, and uterine sensitization-associated gene-1 (USAG-1). Furthermore, BMPs may interact with co-receptors, for example BMP-2 and BMP-4 bind the co-receptor DRAGON (Samad et al. (2005) J. Biol. Chem., 280:14122-9), and extracellular matrix components such as heparin sulfate and heparin (Irie et al. (2003) Biochem. Biophys. Res. Commun., 308:858-865).

### Interactions Between BMPs and Their Antagonists

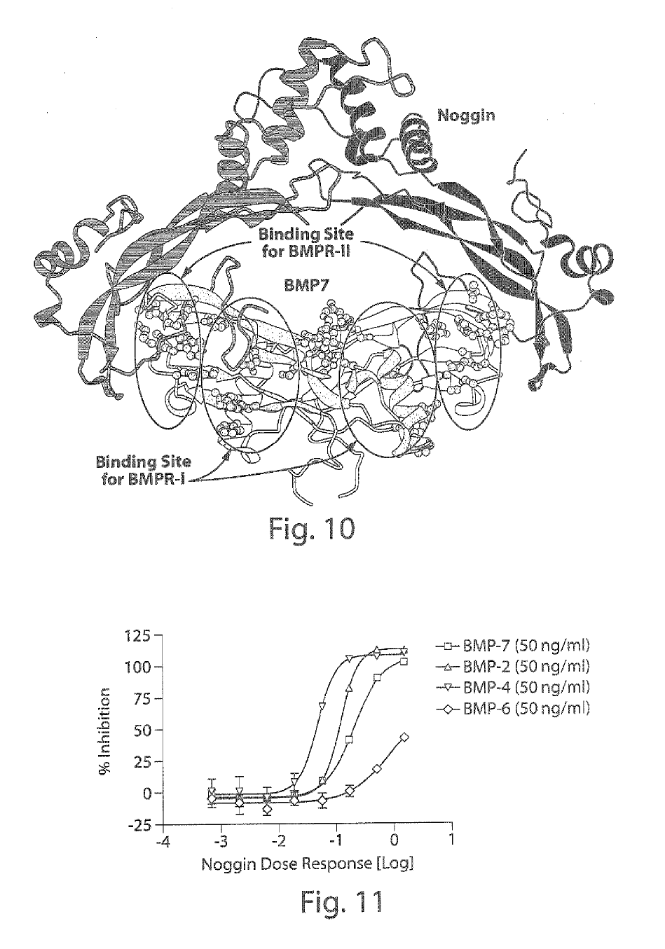

Soluble BMP antagonists like Noggin can bind to BMPs. The Noggin-binding site on BMPs overlaps with both binding sites of type I and type II receptors. For example, a BMP-7-Noggin complex is illustrated in FIG. 10. In addition, Noggin binding to BMP-7 induces a conformational change in BMP-7 that might prevent BMP-7 from binding to and/or activating its receptor. DAN family antagonists, like Noggin, are members of the cysteine knot protein family. Because DAN family proteins and Noggin share this structural motif, they might bind to BMPs in a similar manner. Chordin/SOG antagonists have also been proposed to bind to BMPs in a similar manner. Thus, the present invention contemplates designing a BMP protein able to escape inhibition by Noggin and/or other Noggin-like antagonists.

BMP antagonists like Noggin might be important to eliminate and restrict BMP signaling during development. BMP antagonists might provide spatial regulation through a gradient. Some level of antagonistic action is necessary for proper dorsal-ventral patterning, skeletogenesis and neuronal differentiation in stem cells of the adult subventricular zone of the brain. However, since these antagonists are secreted into the extracellular compartment, their action could extend beyond where they are secreted, which would undesirably decrease the potency of BMP signaling. The present invention contemplates increasing the potency of BMPs by decreasing their susceptibility to inhibition by BMP antagonists such as Noggin. Inhibiting Noggin function might increase the biological activity of BMPs, which could increase bone formation or repair or result in a neurogenic microenvironment in which neuroblasts can proliferate. As used herein, the term “biological activity” refers to any measurable function of BMPs in vivo or in vitro. Some of the ways in which biological activity of BMPs can be measured are listed in the “Examples” section below.

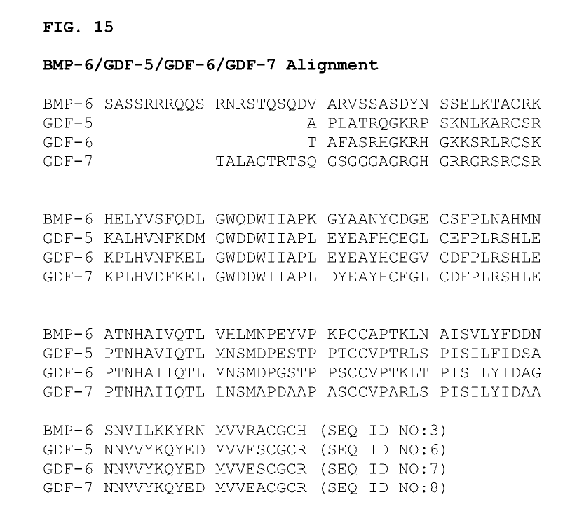

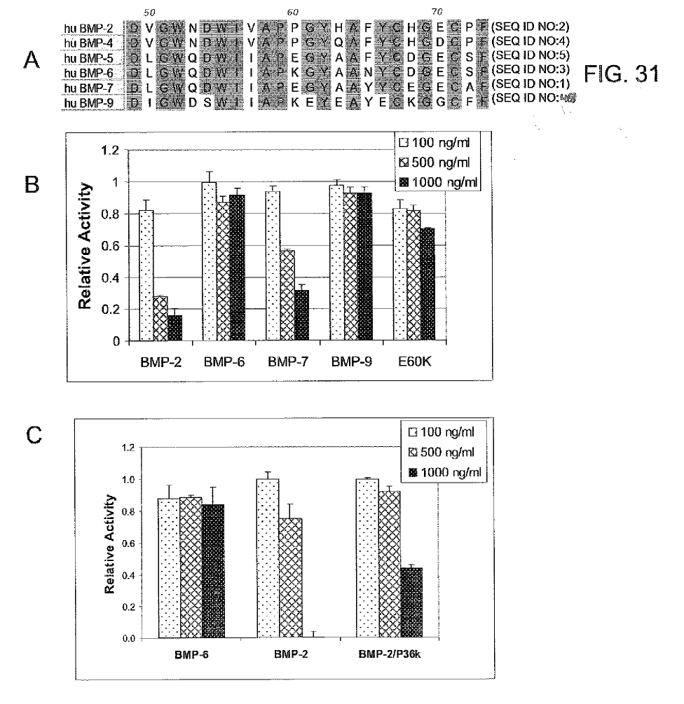

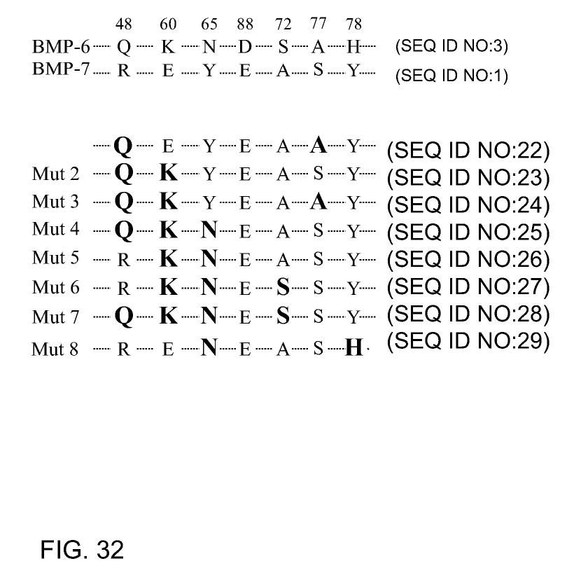

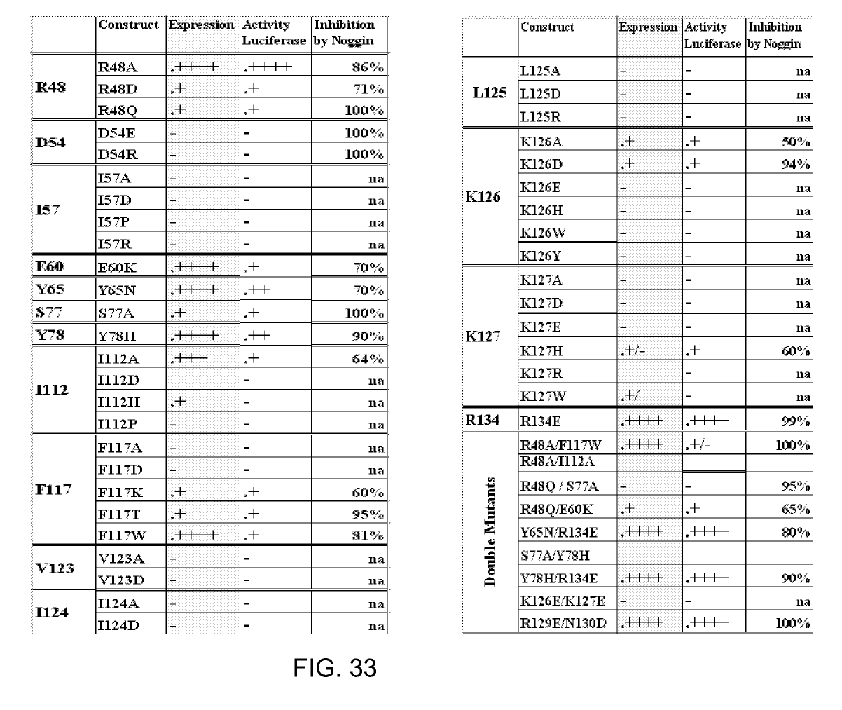

Noggin does not inhibit BMP-6 or BMP-9 to the same degree that it inhibits other BMPs (for example, see FIGS. 11, 12A, 12B and FIG. 31B). A region of BMP-6 important for the reduced susceptibility to Noggin is the region from Val45 to Asn80. When a modified protein is made in which Val45 to Asn80 of BMP-6 replaces the corresponding region of BMP-7, the resulting modified protein is resistant to inhibition by Noggin. It is also contemplated that the region of BMP-9 from Val76 to Thr111 of BMP-9 can also replace the corresponding region of BMP-7 and result in a modified protein that is resistant to inhibition by Noggin. As used herein, the term “corresponding region,” “corresponding portion,” “corresponding amino acid(s),” “corresponding residue(s),” or “corresponding amino acid sequence(s)” refers to the one or more amino acids in a given BMP subfamily member that align with the relevant amino acids in BMP-6 or vice versa, or BMP-9 or vice versa when the two proteins are aligned based on any of a number of alignment algorithms known to those skilled in the art. Also, as used herein, the term “middle region” or “middle portion” or “middle segment” refers to Val45 to Asn80 of BMP-6 or the corresponding region of another BMP subfamily member. Representative alignments for BMP-6 and several BMP subfamily members can be seen in FIGS. 15, 16 and 17. Alignments for a portion of BMP-9 with corresponding portions of other BMP subfamily members can be seen in FIG. 31A.

The middle region of BMP-6 differs from the middle region of BMP-7 by seven amino acids. Therefore, replacing the middle segment of BMP-7 with the corresponding residues in BMP-6 is equivalent to making the following seven amino acid substitutions in BMP-7: R48Q, E60K, Y65N, E68D, A72S, S77A, and Y78H (SEQ ID NO:9). Accordingly, one embodiment of the invention is a modified BMP-7 protein having amino acid substitutions at each of positions R48, E60, Y65, E68, A72, S77, and Y78. In a preferred embodiment, a modified BMP-7 protein contains all of the following amino acid substitutions: R48Q, E60K, Y65N, E68D, A72S, S77A, and Y78H (SEQ ID NO:9).

Additionally, the invention contemplates making BMP mutants with resistance to Noggin inhibition by altering positions in a BMP corresponding to position 48, 60, 65, 68, 72, 77, and 78. As discussed previously, while the substitutions in the mutant may result in the mutant having the same amino acid as the corresponding position in BMP-6, it is also possible according to the invention to substitute in the modified BMP an amino acid at a position corresponding to the position in BMP-6 that is not identical to the amino acid at the corresponding position in BMP-6, but rather that has similar biochemical properties, i.e., that is conservative of the amino acid in BMP-6. For example, if the amino acid in BMP-6 is hydrophobic, according to one embodiment of the invention, it is possible to substitute a different but still hydrophobic amino acid at the corresponding position in the mutant, or if the BMP-6 residue is hydrophillic, to substitute a different but still hydrophilic amino acid, for example.

In another embodiment of the invention, a modified BMP-7 protein has substitutions at one, two, three, four, five, or six of positions R48, E60, Y65, E68, A72, S77, and Y78.

For example, the present invention contemplates a modified BMP-7 having a substitution at least one of the following positions: R48, E60, Y65, E68, A72, S77, and Y78. In one embodiment, the modified BMP-7 has an amino acid substitution at position R48. For example, the modification may be a substitution of a Q, N, W, Y, or F residue in place of the R residue at position 48. In another embodiment, the modified BMP-7 has an amino acid substitution at position E60. For example, the modification may be substitution of a K, R, H, N, or Q residue in place of the E residue at position 60. In another embodiment, the modified BMP-7 has an amino acid substitution at position Y65. For example, the modification may be substitution of an N, Q, H, K, or R residue in place of the Y residue at position 65. In another embodiment, the modified BMP-7 has an amino acid substitution at position E68. In another embodiment, the modified BMP-7 has an amino acid substitution at position A72. For example, the modification may be a substitution of an S, T, Y, N, or Q residue in place of the A residue at position 72. In another embodiment, the modified BMP-7 has an amino acid substitution at position S77. In another embodiment, the modified BMP-7 has an amino acid substitution at position S78.

In another embodiment, the present invention contemplates a modified BMP-7 having at least one of the following amino acid substitutions: R48Q, E60K, Y65N, E68D, A72S, S77A, and Y78H or a substitution at one or more of these positions by an amino acid conservative of the corresponding amino acid in BMP-6.

In one embodiment, the modified BMP-7 has the amino acid substitution R48Q. In one embodiment, the modified BMP-7 has the amino acid substitution E60K. In another embodiment, the modified BMP-7 has the amino acid substitution Y65N. In another embodiment, the modified BMP-7 has the amino acid substitution E68D. In another embodiment, the modified BMP-7 has the amino acid substitution A72S. In another embodiment, the modified BMP-7 has the amino acid substitution S77A. In another embodiment, the modified BMP-7 has the amino acid substitution Y78H. According to another embodiment of the invention, a modified BMP-7 may include one or more substitutions at positions R48, I57, I112, F117, V123, I124, L125, K126, K127, K129, N130, or R134 or a substitution at one or more of these positions by an amino acid conservative of the corresponding amino acid in BMP-6.

Additionally, a modified BMP-7 may include substitutions at two, three, four, five, six, seven, eight, nine, ten, eleven, or all twelve of the foregoing positions.

For example, in one embodiment of the invention, a modified BMP-7 may include one of the following substitutions: R48A, R48D, or R48N.

According to another embodiment of the invention, a modified BMP-7 may include a substitution at position 54. For example, the modification may be one of D54E or D54R.

According to another embodiment of the invention, a modified BMP-7 may include a substitution at position 57. For example, the modification may be one of I57A, I57D, I57P, or I57R.

According to another embodiment of the invention, a modified BMP-7 may include a substitution at position 112. For example, the modification may be one of I112A, I112H, I112D, or I112P.

According to another embodiment of the invention, a modified BMP-7 may include a substitution at position 117. For example, the modification may be one of F117A, F117D, F117K, F117T, or F117W.

According to another embodiment of the invention, a modified BMP-7 may include a substitution at positions 123. For example, the modification may be one of V123 or V123D.

According to another embodiment of the invention, a modified BMP-7 may include a substitution at position 124. For example, the modification may be one of I124A or I124D.

According to another embodiment of the invention, a modified BMP-7 may include a substitution at position 125. For example, the modification may be one of L125A, L125D, or L125R.

According to another embodiment of the invention, a modified BMP-7 may include a substitution at position 126. For example, the modification may be one of K126A, K126D, K126H, K126E, K126Y, or K126W.

According to another embodiment of the invention, a modified BMP-7 may include a substitution at position 127. For example, the modification may be one of K127A, K127D, K127H, K127W, K127E, and K127R

According to another embodiment of the invention, a modified BMP-7 may include a substitution at position 129. For example, the modification may be K129E.

According to another embodiment of the invention, a modified BMP-7 may include a substitution at position 130. For example, the modification may be N130D.

According to another embodiment of the invention, a modified BMP-7 may include a substitution at position 124. For example, the modification may R134E.

The present invention contemplates a modified BMP-7 in which substitutions are made at least two of positions R48, E60, Y65, E68, A72, S77, and Y78. For example, in one embodiment, a modified BMP-7 has substitutions at positions R48 and E60. In another embodiment, a modified BMP-7 has substitutions at position R48 and S77. In another embodiment, a modified BMP-7 has substitutions at positions E60 and Y65. In another embodiment, a modified BMP-7 has substitutions at positions Y65 and Y78. In another embodiment, a modified BMP-7 has substitutions at positions S77 and Y78. In another embodiment, a modified BMP-7 has substitutions at positions Y65 and A72. In another embodiment, a modified BMP-7 has substitutions at positions E60 and A72. In another embodiment, a modified BMP-7 has substitutions at positions R48 and A72.

In another embodiment, the present invention contemplates a modified BMP-7 having at least two of the following amino acid substitutions: R48Q, E60K, Y65N, E68D, A72S, S77A, and Y78H, or a substitution at one or more of these positions by an amino acid conservative of the corresponding amino acid in BMP-6. For example, in one embodiment, a modified BMP-7 has the modifications R48Q and S77A. In another embodiment, a modified BMP-7 has the modifications R48Q and E60K. In another embodiment, a modified BMP-7 has the modifications E60K and Y65N. In another embodiment, a modified BMP-7 has the modifications Y65N and Y78H. In another embodiment, a modified BMP-7 has the modifications S77A and Y78H. In another embodiment, a modified BMP-7 has the modifications R48A and A72S. In another embodiment, a modified BMP-7 has the modifications E60K and A72S. In yet another embodiment, a modified BMP-7 has the modifications Y65N and A72S.

In a further embodiment of the invention, a modified BMP-7 has substitutions at two or more of the following positions: R48, I57, E60, Y65, E68, A72, S77, Y78, I112, F117, V123, I124, L125, K126, K127, K129, N130, and R134. For example, in one embodiment, a modified BMP-7 has substitutions at each of positions R48 and F117. In a particular embodiment, a modified BMP-7 has the modifications R48A and F117W. For example, in one embodiment, a modified BMP-7 has substitutions at each of positions R48 and I112. In a particular embodiment, a modified BMP-7 has the modifications R48A and I112A. For example, in one embodiment, a modified BMP-7 has substitutions at each of positions Y65 and R134. In a particular embodiment, a modified BMP-7 has the modifications Y65N and R134E. For example, in one embodiment, a modified BMP-7 has substitutions at each of positions Y78 and R134. In a particular embodiment, a modified BMP-7 has the modifications Y78H and R134E. For example, in one embodiment, a modified BMP-7 has substitutions at each of positions K126 and K127. In a particular embodiment, a modified BMP-7 has the modifications K126E and K127E. For example, in one embodiment, a modified BMP-7 has substitutions at each of positions R129 and N130. In a particular embodiment, a modified BMP-7 has the modifications R129E and N130D.

The present invention also contemplates a modified BMP-7 in which amino acid substitutions are made at three or more of positions R48, E60, Y65, E68, A72, S77, and Y78. For example, in one embodiment, a modified BMP-7 has substitutions at each of positions E60, Y65, and A72. In another embodiment, a modified BMP-7 has substitutions at each of positions R48, E60, and Y65.

The present invention contemplates a modified BMP-7 in which at least three of the following amino acids substitutions are made: R48Q, E60K, Y65N, E68D, A72S, S77A, and Y78H, or a substitution at one or more of these positions by an amino acid conservative of the corresponding amino acid in BMP-6. In a particular embodiment, a modified BMP-7 includes the substitutions E60K, Y65N, and A72S. In another embodiment, a modified BMP-7 includes the substitutions R48Q, E60K, and Y65N. In yet another embodiment, a modified BMP-7 includes the substitutions R48Q, E60K, and S77A.

The present invention also contemplates a modified BMP-7 in which amino acid substitutions are made at four or more of positions R48, E60, Y65, E68, A72, S77, and Y78. For example, in one embodiment, a modified BMP-7 has substitutions at each of positions R48, E60, Y65, and A72. For example, at position R48, a Q, N, W, Y, or F residue is substituted in place of the R residue. For example, at position E60, a K, R, N, H, or Q residue is substituted in place of the E residue. For example, at position Y65, an N, Q, H, K, or R residue is substituted in place of the Y residue. For example, at position A72, an S, T, Y, N, or Q residue is substituted in place of the A residue.

The present invention contemplates a modified BMP-7 in which at least four of the following amino acid substitutions are made: R48Q, E60K, Y65N, E68D, A72S, S77A, and Y78H, or a substitution at one or more of these positions by an amino acid conservative of the corresponding amino acid in BMP-6. For example, in one embodiment, a modified BMP-7 includes the substitutions R48Q, E60K, Y65N, and A72S.

The present invention further contemplates a modified BMP-7 in which amino acid substitutions are made at five, six, or all seven of the following positions: R48, E60, Y65, E68, A72, S77, and Y78. In one embodiment, substitutions are made at each of the seven position. In a further embodiment, a modified BMP-7 contains five, six, or all seven of the following amino acid substitutions: R48Q, E60K, Y65N, E68D, A72S, S77A, and Y78H, or a substitution at one or more of these positions by an amino acid conservative of the corresponding amino acid in BMP-6.

Because BMP subfamily members are conserved both in sequence and in function, it is expected that replacing the middle region of a given BMP subfamily member with the corresponding region of BMP-6 will confer resistance to Noggin. The present invention contemplates designing modified proteins that are resistant to inhibition by Noggin or a Noggin-like protein, in which one or more of the amino acid sequences in the region from Val45 to Asn80 of BMP-6 replaces one or more amino acid sequences in the corresponding region of any BMP subfamily member.

For example, the present invention contemplates a modified BMP-2 comprising an amino acid substitution at least one of the following positions: D22, S24, V26, N29, V33, P36, H39, F41, H44, P48, A52, D53 and/or L55. In one embodiment, the modified BMP-2 contains a modification at each of the foregoing positions. In a preferred embodiment, the modified BMP-2 contains substitutions at each of S24, P36, F41, H44, P48, and D53. In a preferred embodiment, the modified BMP-2 contains substitutions at one or more of the following positions S24, P36, F41, H44, P48, and D53. In another preferred embodiment, the modified BMP-2 contains a substitution at P36.

The present invention also contemplates a modified BMP-2 comprising at least one of the following amino acid substitutions: D22S, S24Q, V26L, N29Q, V33I, P36K, H39A, F41N, H44D, P48S, A52N, D53A and/or L55M, or a substitution at one or more of these positions by an amino acid conservative of the corresponding amino acid in BMP-6. In one embodiment, the modified BMP-2 contains all of the foregoing amino acid substitutions. In a preferred embodiment, the modified BMP-2 contains each of the following amino acid substitutions: S24Q, P36K, F41N, H44D, P48S, and/or D53A. In a preferred embodiment, the modified BMP-2 contains one or more of the following amino acid substitutions: S24Q, P36K, F41N, H44D, P48S, and/or D53A. In another preferred embodiment, the modified BMP-2 contains the amino acid substitution P36K.

In another embodiment, the present invention relates to a modified BMP-4 comprising an amino acid substitution at least one of the following positions: D24, S26, V28, N31, V35, P38, Q41, F43, H46, D48, P50, A54, D55, and L57. In one embodiment, the modified BMP-4 contains a modification at each of the foregoing positions. In a preferred embodiment, the modified BMP-4 comprises an amino acid substitution at one or more of the following positions S26, P38, F43, P50, and A54. In a preferred embodiment, the modified BMP-4 comprises an amino acid substitution at each of the following positions S26, P38, F43, P50, and A54. In another preferred embodiment, the modified BMP-4 contains a substitution at P38.

In another embodiment, the present invention relates to a modified BMP-4 comprising at least one of the following amino acid substitutions: D24S, S26Q V28L, N31Q, V35I, P38K, Q41A, F43N, H46D, D48E, P50S, A54N, D55A, and L57M, or a substitution at one or more of these positions by an amino acid conservative of the corresponding amino acid in BMP-6. In another embodiment, the modified BMP-4 contains all of the foregoing amino acid substitutions. In a preferred embodiment, the modified BMP-4 comprises one or more of the amino acid substitutions S26Q, P38K, F43N, P50S, and/or A54N. In another preferred embodiment, the modified BMP-4 comprises each of the amino acid substitutions S26Q, P38K, F43N, P50S, and/or A54N. In yet another preferred embodiment, the modified BMP-4 contains the substitution P38K.

In another embodiment, the invention relates to modified BMP-5 an amino acid substitution at least one of the following positions: R41, E53, or F58. In a preferred embodiment, the modified BMP-5 contains substitutions at each of positions R41, E53, and F58. In yet another preferred embodiment, the modified BMP-5 contains a substitution at position E53.

In another embodiment, the invention relates to modified BMP-5 comprising at least one of the amino acid substitutions R41Q, E53K, or F58N, or a substitution at one or more of these positions by an amino acid conservative of the corresponding amino acid in BMP-6. In a preferred embodiment, the modified BMP-5 contains substitutions at each of the following amino acid substitutions: R41Q, E53K, or F58N. In yet another preferred embodiment, the modified BMP-5 contains the amino acid substitution E53K.

The present invention also relates to modified Growth Differentiation Factors (GDFs). GDFs are also members of the BMP subfamily.

For example, the present invention contemplates a modified GDF-5 comprising an amino acid substitution at least one of the following positions: N27, K29, M31, D34, L41, E42, E44, F46, H47, E49, L51, E53, R57, S58, L60, and E61. In a preferred embodiment, the modified GDF-5 contains an amino acid substitution at position L41. Further, the present invention contemplates a modified GDF-5 comprising at least one of the following amino acid substitutions: N27S, K29Q, M31L, D34Q, L41K, E42G, E44A, F46N, H47Y, E49D, L51E, E53S, R57N, S58A, L60M, and E61N, or a substitution at one or more of these positions by an amino acid conservative of the corresponding amino acid in BMP-6. In a preferred embodiment, the modified GDF-5 contains the amino acid substitution L41K.

In another embodiment, the invention relates to a modified GDF-6 comprising an amino acid substitution at least one of positions N27, K29, E30, D34, L41, E42, E44, Y46, H47, E48, V51, D53, R57, S58, L60, and E61. In a preferred embodiment, the modified GDF-6 contain an amino acid substitution at position L41. In another embodiment, the invention relates to a modified GDF-6 comprising one or more of the amino acid substitutions N27S, K29Q, E30D, D34Q, L41K, E42G, E44A, Y46N, H47Y, E48D, V51E, D53S, R57N, S58A, L60M, and E61N, or a substitution at one or more of these positions by an amino acid conservative of the corresponding amino acid in BMP-6. In a preferred embodiment, the modified GDF-6 contains the amino acid substitution L41K.

In yet another embodiment, the invention contemplates a modified GDF-7 comprising an amino acid substitution at least one of the following position: D36, K38, E39D, D43, L50, D51, E53, Y55, H56, E58, L60, D62, R66, S67, L69, and E70. In a preferred embodiment, the modified GDF-7 contains an amino acid substitution at position L50. In yet another embodiment, the invention contemplates a modified GDF-7 comprising at least one amino acid substitution selected from the group consisting of D36S, K38Q, E39D, D43Q, L50K, D51G, E53A, Y55N, H56Y, E58D, L60E, D62S, R66N, S67A, L69M, and E70N, or a substitution at one or more of these positions by an amino acid conservative of the corresponding amino acid in BMP-6. In a preferred embodiment, the modified GDF-7 contains the amino acid substitution L50K.

The foregoing embodiments of the invention are not meant to limit the scope of the invention. BMP subfamily members in addition to those specifically mentioned may be similarly modified. Additional amino acid substitutions, deletions or additions may be made. All that is required is that one or more of the amino acid residues in the middle portion of a BMP subfamily member be replaced with the corresponding one or more amino acid residue from BMP-6, such that the resulting protein is not identical to the wild-type BMP subfamily member nor to wild-type BMP-6 and is not a naturally-occurring protein.

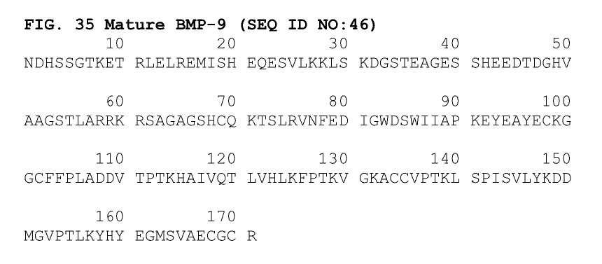

Further, as previously mentioned, because BMP-9 also has resistance to Noggin inhibition in a similar magnitude to that of BMP-6 (see FIG. 31B), it is also contemplated by the invention that BMP mutants be created which correspond with the middle region of BMP-9, i.e., the region of BMP-9 corresponding to residues 45-80 of BMP-6 which are residues Val76 to Thrill of BMP-9 as shown in FIG. 35.

For example, a modified BMP-7 of the invention may include a modification at any one or more of positions S46, R48, L50, Q53, D54, E60, G61, A63, Y66, E68, E70, A72, N76, S77, Y78, M79 and/or N80 of BMP-7. In particular, a modified BMP-7 may include any one or more of the following modifications: S46N, R48E, L50I, Q53D, D54S, E60K, G61E, A63E, Y66E, E68K, E70G, A72F, N76A, S77D, Y78D, M79V, and/or N80T or a substitution of an amino acid at any one of these positions that is conservative of the corresponding amino acid in BMP-9.

For example, a modified BMP-2 of the invention may include a modification at any one or more of positions D22, S24, V26, N29, D30, V33, P36, G37, H38, F40, Y41, H43, E45, P47, H54, L55, and/or N56 of BMP-2. In particular, a modified BMP-2 may include any one or more of the following modifications: D22N, S24E, V26I, N299D, D30S, V33I, P36K, G37E, H38E, F40Y, Y41E, H43K, E45G, P47F, H54D, L55V, and/or N56T or a substitution of an amino acid at any one of these positions that is conservative of the corresponding amino acid in BMP-9.

For example, a modified BMP-4 of the invention may include a modification at any one or more of positions D24, S26, V28, N31, D32, V35, P38, G39, Q41, F43, Y44, H46, D48, P50, H56, L57, and/or N568 of BMP-4. In particular, a modified BMP-4 may include any one or more of the following modifications: D24N, S26E, V28I, N31D, D32S, V35I, P38K, G39E, Q41E, F43Y, Y443, H46K, D48G, P50F, H56D, L57V and/or N58T or a substitution of an amino acid conservative of the corresponding amino acid in BMP-9.

For example, a modified BMP-5 of the invention may include a modification at any one or more of positions S39, R41, L43, Q46, D47, E53, G54, A56, F58, Y59, D61, E63, S65, N69, A70, H71, M72, and/or N73 of BMP-5. In particular, a modified BMP-5 may include any one or more of the following modifications: S39N, R41E, L43I, Q46D, D47S, E53K, G54E, A56E, F58Y, Y59E, D61K, E63G, S65F, N69A, A70D, H71D, M72V, and/or N73T or a substitution at one or more of these positions by an amino acid conservative of the corresponding amino acid in BMP-9.

Modified GDF-5,-6, and -7s according to the invention may also be made that have modifications in their middle regions corresponding to the corresponding amino acid residues in BMP-9 or having substitutions in their middle regions that are conservative of the corresponding amino acids in the middle region of BMP-9.

### Generating Modified BMPs

As used herein, “designed BMPs,” “modified BMPs,” “chimeric BMPs,” “mutant BMPs” and “variant BMPs” are used as equivalents unless stated otherwise. By “designed BMPs” or “modified BMPs” and grammatical equivalents thereof herein is meant non-naturally occurring BMPs which differ from a wild-type or parent BMP by at least one amino acid insertion, deletion, or substitution. It should be noted that unless otherwise stated, all positional numbering of designed or modified BMPs is based on the sequences of the mature native BMPs. Designed BMPs are characterized by the predetermined nature of the variation, a feature that sets them apart from naturally occurring allelic or interspecies variation of the BMP sequence. BMP variants must retain at least 50% of wild type BMP activity in one or more cell types, as determined using an appropriate assay described below. Variants that retain at least 75%, 80%, 85%, 90% or 95% of wild type activity are more preferred, and variants that are more active than wild type are especially preferred. A designed or modified BMP may contain insertions, deletions, and/or substitutions at the N-terminus, C-terminus, or internally. In a preferred embodiment, designed or modified BMPs have at least 1 residue that differs from the most similar human BMP sequence, with at least 2, 3, 4, 5, 6, 7, 8, 9, 10 or more different residues being more preferred.

Further, according to one embodiment of the invention, designed or modified BMPs are not identical to wild-type BMP-6 or wild-type BMP-9.

Designed or modified BMPs of the invention maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the corresponding wild-type BMP protein sequence.

Designed or modified BMPs of the invention may maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the conserved cysteine domain of the C-terminal region of the corresponding wild-type BMP protein sequence.

By “corresponding wild-type protein” it is meant the wild-type version of the modified BMP. For example, if the modified BMP is a modified BMP-7, the corresponding wild-type BMP is wild-type BMP-7.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions×100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used.

Designed or modified BMPs may contain further modifications, for instance mutations that alter additional protein properties such as stability or immunogenicity or which enable or prevent posttranslational modifications such as PEGylation or glycosylation. Modified BMPs may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. By “designed BMP nucleic acids,” “modified BMP nucleic acids,” “chimeric BMP nucleic acids,” “variant BMP nucleic acids,” “mutant BMP nucleic acids” and grammatical equivalents herein is meant nucleic acids that encode designed or modified BMPs. Due to the degeneracy of the genetic code, an extremely large number of nucleic acids may be made, all of which encode the designed or modified BMPs of the present invention, by simply modifying the sequence of one or more codons in a way that does not change the amino acid sequence of the designed BMP. In this application, the use of “or” means “and/or” unless stated otherwise.

As described above, BMPs are naturally expressed as pro-proteins comprising a long pro-domain, one or more cleavage sites, and a mature domain. This pro-protein is then processed by the cellular machinery to yield a dimeric mature BMP molecule. In a preferred embodiment, the modified BMPs of the invention are produced in a similar manner. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et al. (2001) Growth Factors, 18:251-259). Preferably, the modified BMPs of the invention are produced and/or administered therapeutically in this form. Alternatively, BMPs may be produced in other forms, including, but not limited to, mature domain produced directly or refolded from inclusion bodies, or full-length intact pro protein. The modified BMPs of the invention are expected to find use in these and other forms.

In a preferred embodiment, the modified bone morphogenetic protein of the invention is a modified BMP-7, such as a modified or mutant human BMP-7. The amino acid sequence of the native pro-protein of human BMP-7 is shown in FIG. 1A. The amino acid sequence of the native mature human BMP-7 is shown in FIG. 1B. It is to be understood that, although the amino acid sequence of a subunit of the mature dimeric form of human BMP-7 is set forth in FIG. 1B, each subunit can be independently full length or truncated at the N-terminus. For example, a subunit can have any or all of the residues 1 to 37 of the full length mature form as shown in FIG. 1B. Modified BMP nucleic acids and proteins of the invention may be produced using a number of methods known in the art, as elaborated below.

### Preparing Nucleic Acids Encoding Modified BMPs

The invention also includes nucleic acids encoding the modified BMPs described herein. Nucleic acids encoding the modified BMPs described herein can be prepared according to methods known in the art.

In a preferred embodiment, nucleic acids encoding modified BMPs are prepared by total gene synthesis, or by site-directed mutagenesis of a nucleic acid encoding wild type or modified BMPs. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001)).

### Expression Vectors

In a preferred embodiment, an expression vector that comprises the components described below and a gene encoding a modified BMP of the invention is prepared. Numerous types of appropriate expression vectors and suitable regulatory sequences for a variety of host cells are known in the art. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the modified BMP from the host cell. Suitable secretory leader sequences that lead to the secretion of a protein are known in the art. The signal sequence typically encodes a signal peptide comprised of hydrophobic amino acids, which direct the secretion of the protein from the cell. The protein is either secreted into the growth media or, for prokaryotes, into the periplasmic space, located between the inner and outer membrane of the cell. For expression in bacteria, bacterial secretory leader sequences, operably linked to a variant BMP encoding nucleic acid, are usually preferred.

In addition, the cleavage site between the pro-domain and the mature region of the modified BMP of the invention may be modified according to techniques know in the art so that the cleavage site is more efficiently cleaved by proteases such as Furin.

### Transfection/Transformation

The modified BMP nucleic acids are introduced into the cells either alone or in combination with an expression vector in a manner suitable for subsequent expression of the nucleic acid. The method of introduction is largely dictated by the targeted cell type. Exemplary methods include CaPO₄ precipitation, liposome fusion (e.g., using the reagent Lipofectin® or FuGene), electroporation, viral infection (e.g., as outlined in PCT/US97/01019,), dextran-mediated transfection, polybrene mediated transfection, protoplast fusion, direct microinjection, etc. The modified BMP nucleic acids may stably integrate into the genome of the host cell or may exist either transiently or stably in the cytoplasm.

### Hosts for Expression of Modified BMPs

Appropriate host cells for the expression of modified BMPs include yeast, bacteria, archaebacteria, fungi, and insect and animal cells, including mammalian cells. Of particular interest are fungi such as Saccharomyces cerevisiae and Pichia pastoris and mammalian cell lines including 293 (e.g., 293-T and 293-EBNA), BHK, CHO (e.g., CHOK1 and DG44), COS, Jurkat, NIH3T3, etc. (see the ATCC cell line catalog). Modified BMPs can also be produced in more complex organisms, including but not limited to plants (such as corn, tobacco, and algae) and animals (such as chickens, goats, cows); see for example Dove, Nature Biotechnol., 20:777-779 (2002). In one embodiment, the cells may be additionally genetically engineered, that is, contain exogenous nucleic acid other than the expression vector comprising the modified BMP nucleic acid.

### Expression Methods

The modified BMPs of the present invention are produced by culturing a host cell transformed with an expression vector containing nucleic acid encoding a modified BMP, under the appropriate conditions to induce or cause expression of the modified BMP. Either transient or stable transfection methods may be used. The conditions appropriate for modified BMP expression will vary with the choice of the expression vector and the host cell, and will be easily ascertained by one skilled in the art through routine experimentation.

### Purification

In a preferred embodiment, the modified BMPs are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a modified BMP may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, N.Y., 3d ed. (1994). The degree of purification necessary will vary depending on the desired use, and in some instances no purification will be necessary.

### Posttranslational Modification and Derivatization

Once made, the modified BMPs may be covalently modified. Covalent and non-covalent modifications of the protein are thus included within the scope of the present invention. Such modifications may be introduced into a modified BMP polypeptide by reacting targeted amino acid residues of the polypeptide with an organic derivatizing agent that is capable of reacting with selected side chains or terminal residues. Optimal sites for modification can be chosen using a variety of criteria, including but not limited to, visual inspection, structural analysis, sequence analysis and molecular simulation. Sites for modification may be located in the pro-domain or the mature domain.

In one embodiment, the modified BMPs of the invention are labeled with at least one element, isotope or chemical compound. In general, labels fall into three classes: a) isotopic labels, which may be radioactive or heavy isotopes; b) immune labels, which may be antibodies or antigens, and c) colored or fluorescent dyes. The labels may be incorporated into the compound at any position. Labels include but are not limited to biotin, tag (e.g., FLAG, Myc) and; fluorescent labels (e.g., fluorescein). Derivatization with bifunctional agents is useful, for instance, for cross linking a modified BMP to a water-insoluble support matrix or surface for use in the method for purifying anti-modified BMP antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-&lsqb;(p-azidophenyl) dithio&rsqb; propioimidate. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group. Such derivatization may improve solubility, absorption, transport across the blood brain barrier, serum half-life, and the like. Modifications of modified BMP polypeptides may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980).

Another type of covalent modification of modified BMP comprises linking the modified BMP polypeptide to one of a variety of non-proteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. A variety of coupling chemistries may be used to achieve PEG attachment, as is well known in the art.

Another type of modification is chemical or enzymatic coupling of glycosides to the modified BMP. Such methods are described in the art, e.g., in WO 87/05330 published 11 Sep. 1987, and in Aplin and Wriston, CRC Crit. Rev. Biochem., pp. 259-306 (1981).

Alternatively, removal of carbohydrate moieties present on the modified BMP polypeptides may be accomplished chemically or enzymatically. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

### Biophysical and Biochemical Characterization of Designed BMPs

The designed BMPs of the invention can be characterized by their responses to inhibitors such as Noggin, using methods known in the art, for example, Biacore methods. The modified BMPs can also be characterized using cell-based and in vivo assays routinely used in evaluating osteogenic and chondrogenic factors, for example, the alkaline phosphatase assay, the osteoblast proliferation assay, the bone nodule assay, the qPCR-based gene expression assay, or the BMP-Response Element (BRE) Luciferase assay. The biophysical and biochemical characterization of modified BMPs are elaborated below.

### Assaying the Activity of Modified BMPs

In preferred embodiments, the activity of the wild-type and modified BMPs are analyzed using in vitro receptor binding assays, cell-based activity assays, or in vivo activity assays.

### Receptor Binding Assays

The effect of Noggin on the ability of modified BMPs to bind to one or more BMP receptors can be determined by receptor binding assays. For example, affinities for ALK-2, ALK-3, ALK-6, ActRII, ActRIIb, or BMPRII can be determined. Suitable binding assays include, but are not limited to, ELISA, fluorescence anisotropy and intensity, scintillation proximity assays (SPA), Biacore (Pearce et al., Biochemistry 38:81-89 (1999)), DELFIA assays, and AlphaScreen™ (commercially available from PerkinElmer; Bosse R., Illy C., and Chelsky D (2002)).

In a preferred embodiment, Biacore or surface plasmon resonance assays are used. See, for example, McDonnell (2001) Curr. Opin. Chem. Biol. 5:572-577. Typically, Biacore experiments may be performed, for example, by binding BMP receptor-Fc fusion proteins to a protein A derivitized chip or an NTA chip and testing each modified BMP as an analyte. It is also possible to bind an anti-BMP antibody to the chip, or to bind the modified BMP to the chip and test soluble receptor or Fc-receptor fusion proteins as analytes. Biacore experiments have been used previously to characterize binding of TGF-β isoforms to their receptors (De Crescenzo et al. (2001) J. Biol. Chem., 276: 29632-29643; De Crescenzo et al. (2003) J. Mol. Biol., 328:1173-1183).

Alternatively, a plate-based Direct Binding Assay is used to determine the affinity of one or more modified BMPs for one or more BMP receptors. This method is a modified sandwich ELISA in which BMP is captured using an anti-BMP monoclonal antibody and then detected using a BMP receptor-Fc fusion protein.

AlphaScreen™ assays (Bosse R. et al. (2002) Principles of AlphaScreen™, PerkinElmer Literature Application Note Ref #4069. http://lifesciences.perkinelmer. com/Notes/S4069-0802.pdf) can be used to characterize receptor and inhibitor binding. AlphaScreen™ is a bead-based non-radioactive luminescent proximity assay where the donor beads are excited by a laser at 680 nm to release singlet oxygen. The singlet oxygen diffuses and reacts with the thioxene derivative on the surface of acceptor beads leading to fluorescence emission at 600 nm. The fluorescence emission occurs only when the donor and acceptor beads are brought into close proximity by molecular interactions occurring when each is linked to ligand and receptor (or ligand and inhibitor) respectively. This interaction may be competed away by adding an appropriate amount of unlabeled modified BMP that binds the relevant receptor or inhibitor.

In one embodiment, AlphaScreen™ assays are performed using 1) native BMP labeled by a first suitable tag or label; 2) donor beads capable of binding the first tag or label; 3) a BMP receptor or inhibitor labeled by a second suitable tag or label; 4) acceptor beads capable of binding the second tag or label, and 5) varying amounts of an unlabeled modified BMP molecule (e.g., a modified BMP-7), which acts as a competitor. It is also possible to coat the donor or acceptor beads with antibodies that specifically recognize the native BMP or BMP receptor, or to bind the receptor to the donor beads and the ligand to the acceptor beads. In an alternate embodiment, AlphaScreen™ assays are performed using 1) a type I BMP receptor labeled by a first suitable tag or label; 2) donor beads capable of binding the first tag or label; 3) a type II BMP receptor labeled by a second suitable tag or label; 4) acceptor beads capable of binding the second tag or label; 5) native BMP, and 6) varying amounts of an unlabeled modified BMP molecule (e.g., a modified BMP-7), which acts as a competitor. It is also possible to bind the type I BMP receptor to the acceptor beads and the type II BMP receptor to the donor beads.

Fluorescence assays can also be used to characterize receptor and inhibitor binding. For example, either BMP-7 or a BMP-7 receptor or inhibitor may be labeled with a fluorescent dye (for examples of suitable dyes, see the Molecular Probes catalog). As is known in the art, the fluorescence intensity or anisotropy of a labeled molecule may change upon binding to another molecule. Fluorescence assays may be performed using 1) fluorescently labeled native BMP (e.g., BMP-7), 2) a BMP receptor or inhibitor, and 3) varying amounts of an unlabeled modified BMP (e.g., modified BMP-7), which acts as a competitor.

Additionally, scintillation proximity assays (SPA) can be used to determine receptor binding affinity. For example, BMP receptor-Fc fusions may be bound to protein A coated SPA beads or flash-plate and treated with S³⁵-labeled BMP; the binding event results in production of light.

### Cell-Based Activity Assays

BMPs promote the growth and differentiation of a number of types of cells. BMP activity may be monitored, for example, by measuring BMP-induced differentiation of human bone marrow-derived mesenchymal stem cells (hMSCs), MC3T3-E1 (an osteoblast-like cell derived from murine calvaria), C3H10T1/2 (a mouse mesenchymal stem cell line derived from embryonic connective tissue), ATDC5 (a mouse embryonic carcinoma cell), L-6 (a rat myoblast cell line) or C2C12 (a mouse myoblastic cell line) cells. Differentiation may be monitored using, for example, luminescence reporters for alkaline phosphatase or calorimetric reagents such as Alcian Blue or PNPP (Layery et al., (2008), JBC, 283:20948-20958; Asahina et al. (1996) Exp. Cell Res., 222:38-47; Inada et al. (1996) Biochem. Biophys. Res. Commun., 222:317-322; Jortikka et al. (1998) Life Sci., 62:2359-2368; Cheng et al. (2003) J. Bone Joint Surgery 95A:1544-1552).

The rat limb bud cartilage differentiation assay may also be used to monitor activity in primary cells. In alternative embodiments, reporter gene or kinase assays may be used. Since BMPs activate the JAK-STAT signal transduction pathway, a BMP responsive cell line containing a STAT-responsive reporter such as GFP or luciferase may be used (Kusanagi et al. (2000) Mol. Biol. Cell., 11:555-565). For example, BMP activity in kidney cells may be determined using cell-based assays; see for example Wang and Hirschberg (2004) J. Biol. Chem., 279:23200-23206.

### Animal Models of BMP Activity

Typically, BMP activities in an animal are measured by bone induction following subcutaneous injection. In a preferred embodiment, the activities of one or more modified BMPs are determined in an animal model of a BMP-responsive disease or condition. For example, animal models of renal disease include, but are not limited to, the rat nephrotoxic serum nephritis model (Zeisberg et al. 2003)), the rat chronic cyclosporine A-induced nephropathy model (Li et al. (2004) Am. J. Physiol. Renal Physiol., 286:F46-57), the mouse unilateral ureteral obstruction model (Schanstra et al. (2003) Thromb. Haemost., 89:735-740), streptozotocin-induced diabetic nephropathy (Taneda et al. (2003) J. Am. Soc. Nephrol., 14:968-980), the anti-thy 1.1 mAb and Habu snake venom induced glomerulonephritis models (Dimmler et al. (2003) Diagn. Mol. Pathol., 12:108-117), and the rat 5/6 remnant kidney model (Romero et al. (1999) Kidney Int., 55:945-955). Animal models of liver disease include, but are not limited to, rat bile duct ligation/scission model (Park et al. (2000) Pharmacol. Toxicol., 87:261-268), CCI4 plus ethanol-induced liver damage (Hall et al. (1991) Hepatology, 12:815-819), dimethylnitrosamine-induced liver cirrhosis (Kondou et al. (2003) J. Hepatol., 39:742-748), and thioacetamide-induced liver damage (Muller et al. (1988) Exp. Pathol., 34:229-236). Animal models of lung disease include, but are not limited to, ovalbumin-induced airway fibrosis (Kenyon et al. (2003) Toxicol. Appl. Pharmacol., 186:90-100), bleomycin-induced lung fibrosis (Izbicki et al. (2002) Int. J. Exp. Pathol., 83:111-119), monocrotaline-induced pulmonary fibrosis (Hayashi et al. (1995) Toxicol. Pathol., 23: 63-71), and selective irradiation (Pauluhn et al. (2001) Toxicology, 161: 153-163). Animal models of neurological disease include, but are not limited to, animal models for Parkinson's disease such as the 6-hydroxydopamine (6-OHDA) hemilesioned rat model and MPTP-induced Parkinson's disease, animal models of ALS such as rats or mice expressing mutant SOD1 (Shibata et al. (2002) Neuropathology, 22:337-349), and animal models of stroke induced by intracortical microinjection of endothelin or quinolinic acid (Gilmour et al. (2004) Behav. Brain Res., 150:171-183) or cerebral artery occlusion (Merchenthaler et al. (2003) Ann. NY Acad. Sci. 1007: 89-100).

### Formulation and Administration

Designed BMPs of the present invention can be formulated for administration to a mammal, preferably a human in need thereof as part of a pharmaceutical composition. The composition can be administered by any suitable means, e.g., parenterally, orally or locally. Where the designed BMPs is to be administered locally, as by injection, to a desired tissue site, or systemically, such as by intravenous, subcutaneous, intramuscular, intraorbital, ophthalmic, intraventricular, intracranial, intracapsular, intraspinal, intracistemal, intraperitoneal, buccal, rectal, vaginal, intranasal or aerosol administration, the composition preferably comprises an aqueous solution. The solution preferably is physiologically acceptable, such that administration thereof to a mammal does not adversely affect the mammal's normal electrolyte and fluid volume balance. The aqueous solution thus can comprise, e.g., normal physiologic saline (0.9% NaCl, 0.15M), pH 7-7.4.

Useful solutions for oral or parenteral systemic administration can be prepared by any of the methods well known in the pharmaceutical arts, described, for example, in “Remington's Pharmaceutical Sciences” (Gennaro, A., ed., Mack Pub., 1990, the disclosure of which is incorporated herein by reference). Formulations can include, for example, polyalkylene glycols such as polyethylene glycol, oils of vegetable origin, hydrogenated naphthalenes, and the like. Formulations for direct administration, in particular, can include glycerol and other compositions of high viscosity. Biocompatible, preferably bioresorbable polymers, including, for example, hyaluronic acid, collagen, tricalcium phosphate, polybutyrate, polylactide, polyglycolide and lactide/glycolide copolymers, may be useful excipients to control the release of the designed BMPs in vivo. Other potentially useful parenteral delivery systems for the present designed BMPs can include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate or deoxycholate, or oily solutions for administration in the form of nasal drops or as a gel to be applied intranasally.

Alternatively, the designed BMPs, including designed OP-1/BMP-7, identified as described herein may be administered orally. For example, liquid formulations of designed BMPs can be prepared according to standard practices such as those described in “Remington's Pharmaceutical Sciences” (supra). Such liquid formulations can then be added to a beverage or another food supplement for administration. Oral administration can also be achieved using aerosols of these liquid formulations. Alternatively, solid formulations prepared using art-recognized emulsifiers can be fabricated into tablets, capsules or lozenges suitable for oral administration.

Optionally, the designed BMPs can be formulated in compositions comprising means for enhancing uptake of the analog by a desired tissue. For example, tetracycline and diphosphonates (bisphosphonates) are known to bind to bone mineral, particularly at zones of bone remodeling, when they are provided systemically in a mammal. Accordingly, such components can be used to enhance delivery of the present designed BMPs to bone tissue. Alternatively, an antibody or portion thereof that binds specifically to an accessible substance specifically associated with the desired target tissue, such as a cell surface antigen, also can be used. If desired, such specific targeting molecules can be covalently bound to the present analog, e.g., by chemical crosslinking or by using standard genetic engineering techniques to create, for example, an acid labile bond such as an Asp-Pro linkage. Useful targeting molecules can be designed, for example, according to the teachings of U.S. Pat. No. 5,091,513.

It is contemplated also that some of the designed BMPs may exhibit the highest levels of activity in vivo when combined with carrier matrices, i.e., insoluble polymer matrices. See for example, U.S. Pat. No. 5,266,683 the disclosure of which is incorporated by reference herein. Currently preferred carrier matrices are xenogenic, allogenic or autogenic in nature. It is contemplated, however, that synthetic materials comprising polylactic acid, polyglycolic acid, polybutyric acid, derivatives and copolymers thereof may also be used to generate suitable carrier matrices. Preferred synthetic and naturally derived matrix materials, their preparation, methods for formulating them with the designed BMPs of the invention, and methods of administration are well known in the art and so are not discussed in detailed herein. See for example, U.S. Pat. No. 5,266,683.

Still further, the present designed BMPs can be administered to the mammal in need thereof either alone or in combination with another substance known to have a beneficial effect on tissue morphogenesis. Examples of such substances (herein, cofactors) include substances that promote tissue repair and regeneration and/or inhibit inflammation. Examples of useful cofactors for stimulating bone tissue growth in osteoporotic individuals, for example, include but are not limited to, vitamin D3, calcitonin, prostaglandins, parathyroid hormone, dexamethasone, estrogen and IGF-I or IGF-II. Useful cofactors for nerve tissue repair and regeneration can include nerve growth factors. Other useful cofactors include symptom-alleviating cofactors, including antiseptics, antibiotics, antiviral and antifungal agents, analgesics and anesthetics.

Modified BMPs preferably are formulated into pharmaceutical compositions by admixture with pharmaceutically acceptable, nontoxic excipients and carriers. As noted above, such compositions can be prepared for systemic, e.g., parenteral, administration, particularly in the form of liquid solutions or suspensions; for oral administration, particularly in the form of tablets or capsules; or intranasally, particularly in the form of powders, nasal drops or aerosols. Where adhesion to a tissue surface is desired, the composition can comprise a fibrinogen-thrombin dispersant or other bioadhesive such as is disclosed, for example, in PCT US91/09275, the disclosure of which is incorporated herein by reference. The composition then can be painted, sprayed or otherwise applied to the desired tissue surface.

The compositions can be formulated for parenteral or oral administration to humans or other mammals in therapeutically effective amounts, e.g., amounts which provide appropriate concentrations of the designed BMPs to target tissue for a time sufficient to induce the desired effect. Preferably, the present compositions alleviate or mitigate the mammal's need for a morphogen-associated biological response, such as maintenance of tissue-specific function or restoration of tissue-specific phenotype to senescent tissues (e.g., osteopenic bone tissue).

As will be appreciated by those skilled in the art, the concentration of the compounds described in a therapeutic composition will vary depending upon a number of factors, including the dosage of the drug to be administered, the chemical characteristics (e.g., hydrophobicity) of the compounds employed, and the route of administration. The preferred dosage of drug to be administered also is likely to depend on such variables as the type and extent of a disease, tissue loss or defect, the overall health status of the particular patient, the relative biological efficacy of the compound selected, the formulation of the compound, the presence and types of excipients in the formulation, and the route of administration. In general terms, the compounds of this invention may be provided in an aqueous physiological buffer solution containing about 0.1 to 10% w/v compound for parenteral administration. Typical doses ranges are from about 10 ng/kg to about 1 g/kg of body weight per day; with a preferred dose range being from about 0.1 mg/kg to 100 mg/kg of body weight.

### Therapeutic Uses

The modified BMPs of this invention are capable of inducing the developmental cascade of bone and cartilage morphogenesis. Accordingly, the modified BMPs of the invention may be used to induce proliferation of bone and cartilage in a variety of locations in the body. For example, repair of joints such as knee, elbow, ankle, and finger joints are contemplated by the invention. For example, the modified BMPs of the invention are indicated for regenerating cartilage in patients suffering from arthritis or other cartilage degenerating diseases. Further, the modified BMPs of the invention are indicated for treating tears in cartilage due to injury. In addition, the modified BMPs of the invention are useful for inducing bone growth in patients. For example, the modified BMPs of the invention are indicated for use in treating patients suffering from bone fractures or breaks, osteoporosis, or patients in need of spinal fusion or for repair of the spine, vertebrae or the like.

The modified BMPs of this invention are capable of inducing the developmental cascade of bone morphogenesis and tissue morphogenesis for a variety of tissues in mammals different from bone or bone cartilage. This morphogenic activity includes the ability to induce proliferation and differentiation of progenitor cells, and the ability to support and maintain the differentiated phenotype through the progression of events that results in the formation of bone, cartilage, non-mineralized skeletal or connective tissues, and other adult tissues.

For example, the modified BMPs of the present invention may be used for treatment to prevent loss of and/or increase bone mass in metabolic bone diseases. General methods for treatment to prevent loss of and/or increase bone mass in metabolic bone diseases using osteogenic proteins are disclosed in U.S. Pat. No. 5,674,844, the disclosures of which are hereby incorporated by reference. The modified BMPs of the present invention may also be administered to replace or repair bone or cartilage at injury sites such as bone breaks, bone fractures, and cartilage tears. The modified BMPs of the present invention may be used for periodontal tissue regeneration. General methods for periodontal tissue regeneration using osteogenic proteins are disclosed in U.S. Pat. No. 5,733,878, the disclosures of which are hereby incorporated by reference. The modified BMPs of the present invention may be used for liver regeneration. General methods for liver regeneration using osteogenic proteins are disclosed in U.S. Pat. No. 5,849,686, the disclosures of which are hereby incorporated by reference. The modified BMPs of the present invention may be used for treatment of chronic renal failure. General methods for treatment of chronic renal failure using osteogenic proteins are disclosed in U.S. Pat. No. 6,861,404, the disclosures of which are hereby incorporated by reference. The modified BMPs of the present invention may be used for enhancing functional recovery following central nervous system ischemia or trauma. General methods for enhancing functional recovery following central nervous system ischemia or trauma using osteogenic proteins are disclosed in U.S. Pat. No. 6,407,060, the disclosures of which are hereby incorporated by reference. The modified BMPs of the present invention may be used for inducing dendritic growth. General methods for inducing dendritic growth using osteogenic proteins are disclosed in U.S. Pat. No. 6,949,505, the disclosures of which are hereby incorporated by reference. The modified BMPs of the present invention may be used for inducing neural cell adhesion. General methods for inducing neural cell adhesion using osteogenic proteins are disclosed in U.S. Pat. No. 6,800,603, the disclosures of which are hereby incorporated by reference. The modified BMPs of the present invention may be used for treatment and prevention of Parkinson's disease. General methods for treatment and prevention of Parkinson's disease using osteogenic proteins are disclosed in U.S. Pat. No. 6,506,729, the disclosures of which are hereby incorporated by reference. It is within skills of an ordinary artisan to modify the general methods using the modified BMPs of the present invention for various therapeutic uses described above. Exemplary embodiments of therapeutic applications of the modified BMPs of the present invention are further described below.

The modified BMPs of this invention may be used to repair diseased or damaged mammalian tissue. The tissue to be repaired is preferably assessed, and excess necrotic or interfering scar tissue removed as needed, by surgical, chemical, ablating or other methods known in the medical arts. The modified BMPs then may be provided directly to the tissue locus as part of a sterile, biocompatible composition, either by surgical implantation or injection. Alternatively, a sterile, biocompatible composition containing modified BMP-stimulated progenitor cells may be provided to the tissue locus. The existing tissue at the locus, whether diseased or damaged, provides the appropriate matrix to allow the proliferation and tissue-specific differentiation of progenitor cells. In addition, a damaged or diseased tissue locus, particularly one that has been further assaulted by surgical means, provides a morphogenically permissive environment. For some tissues, it is envisioned that systemic provision of the modified BMPs will be sufficient.

In some circumstances, particularly where tissue damage is extensive, the tissue may not be capable of providing a sufficient matrix for cell influx and proliferation. In these instances, it may be necessary to provide the modified BMPs or modified BMP-stimulated progenitor cells to the tissue locus in association with a suitable, biocompatible formulated matrix, prepared by any of the means described below. The matrix preferably is tissue-specific, in vivo biodegradable, and comprises particles having dimensions within the range of 70-850 μm, most preferably 150-420 μm.

The modified BMPs of this invention also may be used to prevent or substantially inhibit scar tissue formation following an injury. If a modified BMP is provided to a newly injured tissue locus, it can induce tissue morphogenesis at the locus, preventing the aggregation of migrating fibroblasts into non-differentiated connective tissue. The modified BMP preferably is provided as a sterile pharmaceutical preparation injected into the tissue locus within five hours of the injury.

For example, the modified BMPs may be used for protein-induced morphogenesis of substantially injured liver tissue following a partial hepatectomy. Variations on this general protocol may be used for other tissues. The general method involves excising an essentially nonregenerating portion of a tissue and providing the modified BMP, preferably as a soluble pharmaceutical preparation to the excised tissue locus, closing the wound and examining the site at a future date. Like bone, liver has a potential to regenerate upon injury during post-fetal life.

As another example, the modified BMPs of this invention can also be used to induce dentinogenesis. To date, the unpredictable response of dental pulp tissue to injury is a basic clinical problem in dentistry. Using standard dental surgical procedures, small areas (e.g., 2 mm) of dental pulps can be surgically exposed by removing the enamel and dentin immediately above the pulp (by drilling) of sample teeth, performing a partial amputation of the coronal pulp tissue, inducing hemostasis, application of the pulp treatment, and sealing and filling the cavity by standard procedures.

As another example, the modified BMP-induced regenerative effects on central nervous system (CNS) repair may be assessed using a rat brain stab model. Briefly, male Long Evans rats are anesthetized and the head area prepared for surgery. The calvariae is exposed using standard surgical procedures and a hole drilled toward the center of each lobe using a 0.035K wire, just piercing the calvariae. 25 μl solutions containing either a modified BMP or PBS then is provided to each of the holes by Hamilton syringe. Solutions are delivered to a depth approximately 3 mm below the surface, into the underlying cortex, corpus callosum and hippocampus. The skin then is sutured and the animal allowed to recover.

Three days post surgery, rats are sacrificed by decapitation and their brains processed for sectioning. Scar tissue formation is evaluated by immunofluoresence staining for glial fibrillary acidic protein, a marker protein for glial scarring, to qualitatively determine the degree of scar formation.

### EXAMPLES

### Example 1

### BMP Induction of Alkaline Phosphatase Activity

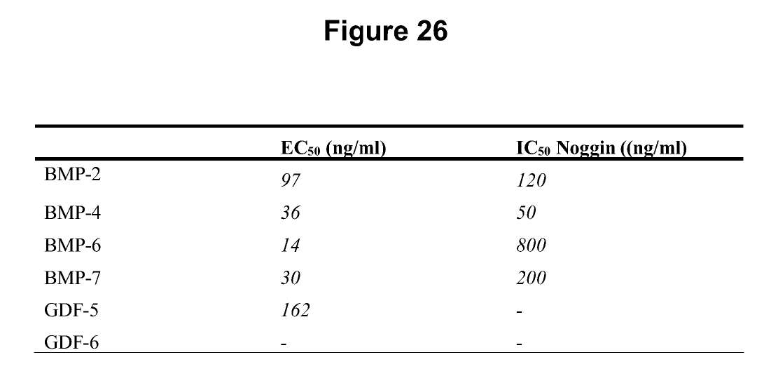

The ability of BMP-2, BMP-4, BMP-5, BMP-6, BMP-7, GDF-5, and GDF-6 to induce alkaline phosphatase (ALP) activity in the rat osteosarcoma cell line ROS17/2.8 was assayed. Each growth factor was tested in a nine point dose response in triplicate. In particular, ROS17/2.8 cells were plated in 96-well tissue culture plates. BMP/GDF were added to the cells in the following dosages: 6000, 2000, 666, 222, 74, 24, 8, 2, and 0.9 ng/ml and incubated for a period of 48 hours. Cells were subsequently lysed and potency of the growth factors to induce ALP activity was assessed based on the EC50 derived from non-linear regression of the mean optical density (OD) of the samples (See FIG. 26).

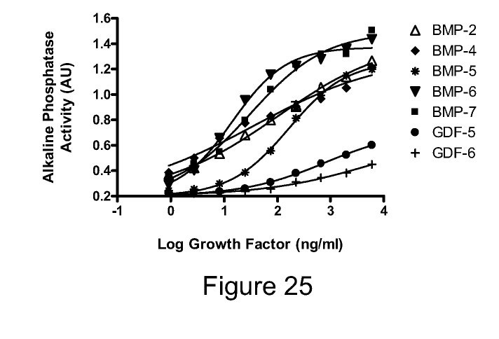

As shown in FIG. 25, all of the BMPs tested demonstrated robust activities, whereas GDF-5 and GDF-6 were significantly less active. Among the growth factors tested, BMP-6 showed the highest potency in inducing alkaline phosphatase activity, followed by BMP-7, BMP-4, BMP-2, BMP-5, GDF-5, and GDF-6 (highest to lowest potency).

### Example 2

### Noggin Inhibition of a Panel of Exemplary BMPs and Related Proteins

BMP inhibition by Noggin was initially assessed using an art-recognized alkaline phosphatase based assay in ROS 17/2.8 cells. Briefly, ROS 17/2.8 cells were plated in 96-well tissue culture plates. BMP-2, -4, -6 and -7 were mixed with increasing concentrations of Noggin and incubated at room temperature for 30 minutes. This mixture was later added to ROS cells so that the final concentration of each BMP was 50 ng/ml. Assays were performed in triplicate. Control wells consisted of cells treated with each BMP alone in the absence of Noggin. Cells were incubated for 48 hours post-treatment. Cells were subsequently lysed and the total BMP-induced alkaline phosphatase activity measured according to standard protocols. The susceptibility of each BMP to Noggin was reported as a percent inhibition. For each of the Noggin concentrations tested, the percent inhibition was calculated according to the following formula:

% inhibition=((AP control−AP Noggin)/AP control)×100

where “AP control” is the mean (n=3) alkaline phosphatase activity in the control wells without Noggin; and “AP Noggin” is the mean (n=3) alkaline phosphatase activity in wells treated with the indicated concentration of Noggin.

Noggin dose response curves as shown in FIG. 11 were derived by fitting the data to a non-linear regression. The IC₅₀ for Noggin corresponding to each BMP assayed was then calculated and the results are shown in FIG. 11. These results indicate that the four BMPs tested are differentially inhibited by Noggin. BMP-4 is the most susceptible to Noggin inhibition, followed by BMP-2, BMP-7, and then BMP-6 being the least susceptible (see FIGS. 11 and 26). While BMP-4, BMP-2, and BMP-7 are clearly sensitive to Noggin inhibition, BMP-6 demonstrated a marked resistance to Noggin inhibition with an IC₅₀ four to sixteen times higher than that of the other BMPs tested and was the least susceptible to Noggin inhibition (See FIGS. 11 and 26). The data were reproducible at both 48 and 144 hrs.

These data were confirmed in a BMP-induced routine reporter gene luciferase assay (RGA) using A549-BRE cells. Briefly, the susceptibility of BMPs to Noggin inhibition was tested using A549-BRE cells which contain a firefly luciferase gene driven by the BMP response element, BRE. A549-BRE cells were seeded into 96-well tissue culture plates in F12K medium with 1% FBS. BMP-2, -4, -6 and -7 were mixed with increasing concentrations of Noggin and incubated at room temperature for 30 minutes. This mixture was later added to A549-BRE cells so that the final concentration of each BMP was 150 ng/ml. Control wells consisted of cells treated with each BMP alone in the absence of Noggin. Cells were incubated for 24 hours post treatment. Cells were subsequently lysed and the total BMP-induced luciferase activity measured using Bright Glo reagent (Promega) according to the manufacturer's recommendations. The susceptibility of each BMP to Noggin inhibition was reported as percent inhibition. For each Noggin concentration tested, the percent inhibition was calculated according to the following formula:

% inhibition=((Luc Control−Luc Noggin)/Luc control)×100

where “Luc Control” is the mean (n=3) luciferase activity in the control wells without Noggin; and “Luc Noggin” is the mean (n=3) luciferase activity in wells treated with the indicated concentration of Noggin.

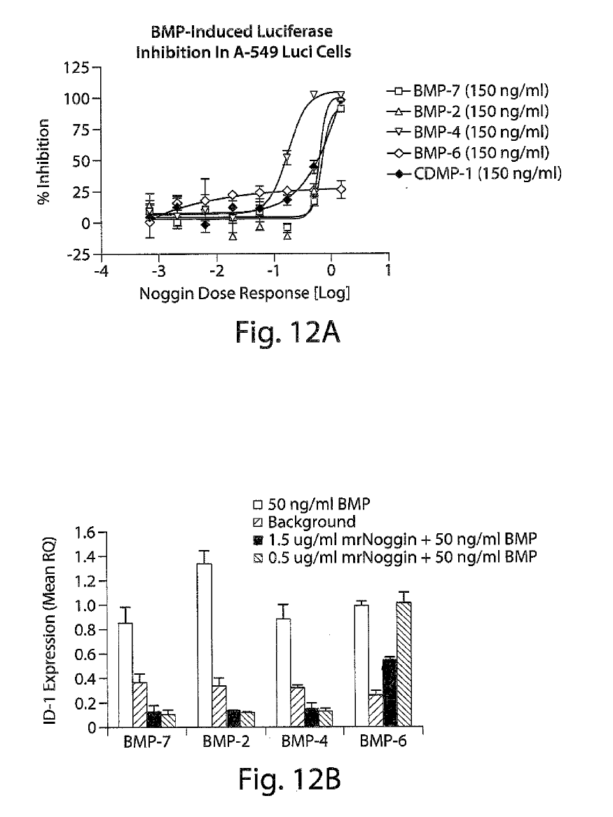

In this assay, the maximum inhibition achieved in the presence of 150 ng/ml of BMP-6 was around 25% (FIG. 12A).

This assay confirmed that of the BMPs tested, BMP-6 is the least susceptible to inhibition by Noggin. Given that this result was obtained in both ROS 17.8 cells and in A549-BRE cells, BMP-6 resistance to noggin inhibition is not restricted to a particular cell type or assay system, but rather is an intrinsic characteristic of BMP-6.

Similar data were also obtained in hMSCs upon measuring BMP-induced ID-1 gene expression by QPCR (FIG. 12B) using routine materials and methods. In this assay, the susceptibility of BMPs to Noggin inhibition was tested using human bone marrow-derived mesenchymal stem cells (hMSC). In brief, hMSCs were seeded onto 48-well tissue culture plates. BMP-2, -4, -6 and -7 were mixed with either 0.5 ug/ml or 1/5 microg/ml of Noggin and incubated at room temperature for 30 minutes. This mixture was later added to hMSC cells so that the final concentration of each BMP is 50 ng/ml. Positive control wells consisted of cells treated with each BMP alone in the absence of Noggin. Negative control wells, also referred to as background, consisted of cells treated with vehicle alone. Cells were incubated for 24 hrs post-treatment. Cells were subsequently lysed, the polyA RNA isolated, and cDNA synthesized. The induction of ID-1 transcript by BMPs was measured by quantitative PCR using 7900HT Real-Time PCR System (Applied BioSystems) according to the manufacturer's protocol. A standard delta/delta Ct method was used for data analysis.

As shown in FIG. 12B, ID-1 gene expression in the presence of Noggin and BMP-2, -4, and -7 was significantly reduced as compared to ID-1 gene expression in the presence of BMP-2, -4, and -7 alone. In contrast, the level of ID-1 gene expression in the presence of 0.5 ug/ml of Noggin and BMP-6 was practically the same as in the presence of BMP-6 alone. Even in the presence of 1.5 ug/ml of Noggin and BMP-6, while ID-1 gene expression was reduced over the lower concentration of Noggin, the level of expression was still over twice the level of background expression of ID-1 in the control.

### Example 3

### BMP-6 Induction of Downstream Genes in Primary Human Bone Marrow-Derived Mesenchymal Stem Cells is Less Susceptible to Noggin Inhibition

The effects of noggin on signaling events after BMP-6 stimulation in primary human bone marrow-derived mesenchymal stem cells (hMSCs) was tested. Upon stimulation by BMPs, hMSCs initiate a signaling cascade involving the oligomerization of type 1 and type 2 receptors and phosphorylation of Smads 1/5/8 that leads to modulation of transcription of BMP target genes.

The levels of transcripts of six BMP target genes including Id-1, Dlx-5, Sp-7, Msx-2, ALP, and noggin itself were compared by quantitative polymerase chain reaction (qPCR) in cells stimulated by either BMP-6 or BMP-7 in the presence or absence of noggin according to standard protocols. In particular, hMSCs were seeded onto 48-well tissue culture plates. BMP-6 and BMP-7 were incubated with 1 ug/mL Noggin at room temperature for 30 minutes. This mixture was then added to hMSC cultures to a final concentration of 50 ng/mL of BMP. Positive control wells consisted of cells treated with each BMP alone in the absence of Noggin. Negative control wells consisted of untreated cells and cells treated with Noggin alone. Cells were incubated for 24 hours post-treatment. Cells were subsequently lysed, the polyA RNA isolated and cDNA synthesized.

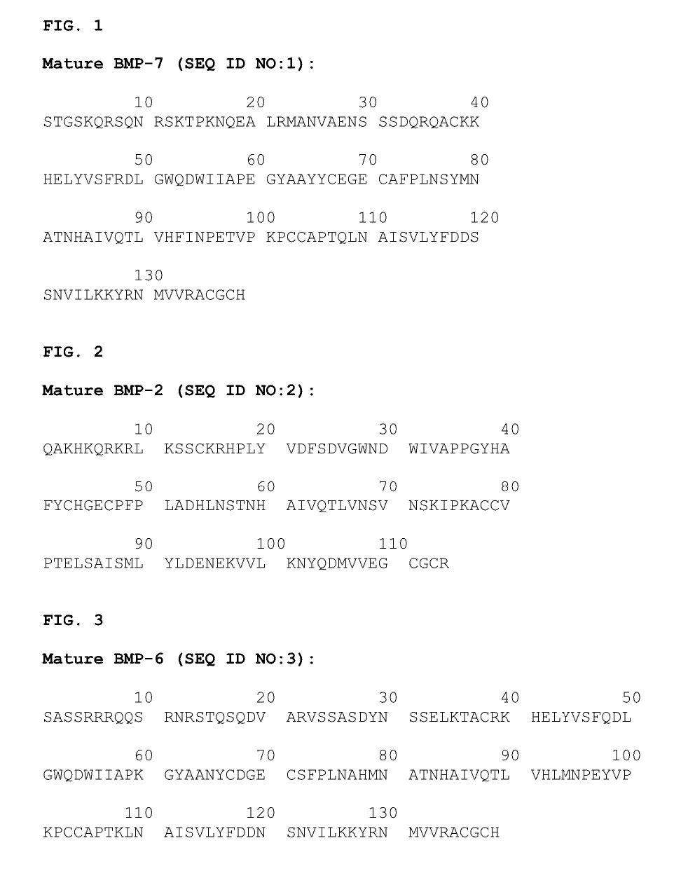

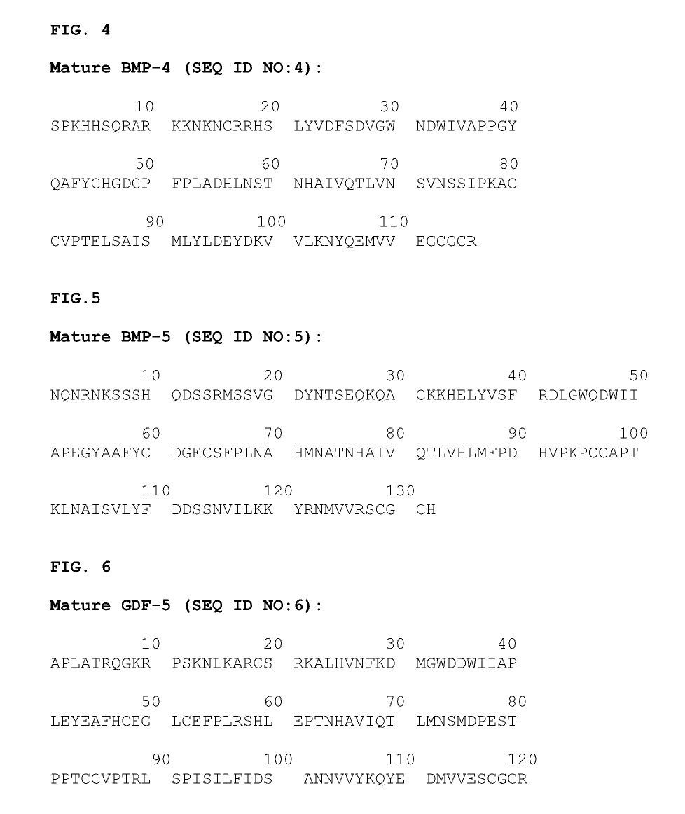

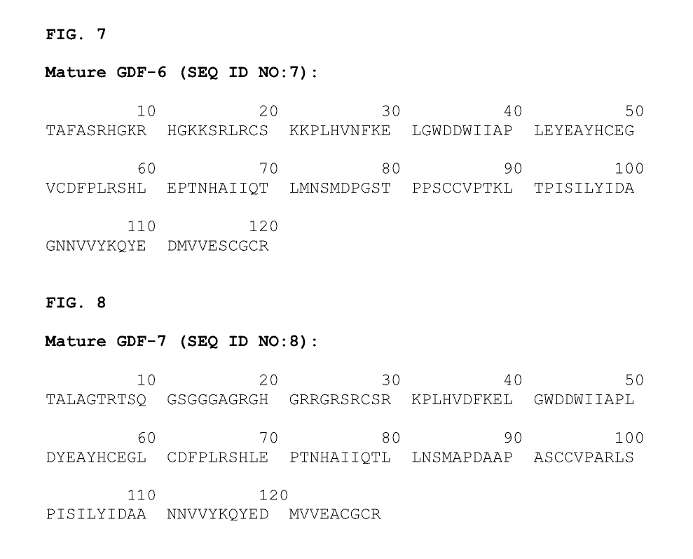

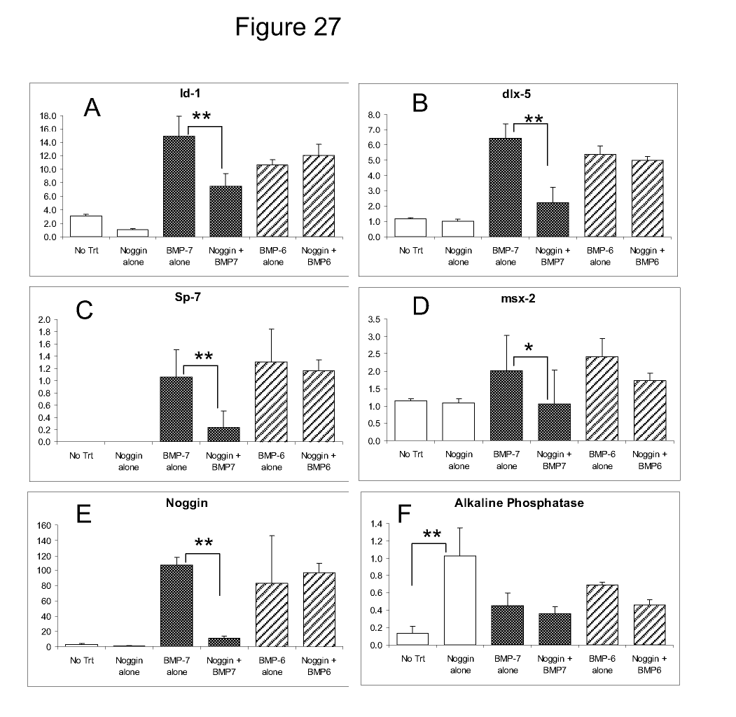

Modulation of gene expression of Id-1, dlx-5, Sp-7, msx-2, ALP, and Noggin following BMP-6 or BMP-7 treatment was assessed in the presence or absence of noggin and results are shown in FIGS. 27A-F. For the target genes Id-1, dlx-5, Sp-7, msx-2, and noggin, BMP-7 mediated induction of transcription was significantly inhibited by noggin (FIGS. 27A-E). In contrast, noggin did not significantly affect induction of Id-1, Dlx-5, Sp-7 and noggin transcription induced by BMP-6 (FIGS. 2A-C, and E). Interestingly, hMSC treatment with noggin, in the absence of any BMP, resulted in the induction of ALP gene expression (FIG. 2). This effect was antagonized by either BMP-6 or BMP-7 (FIG. 2F).

### Example 4

### BMP-7 Variant Proteins with Marked Resistance to Noggin Inhibition

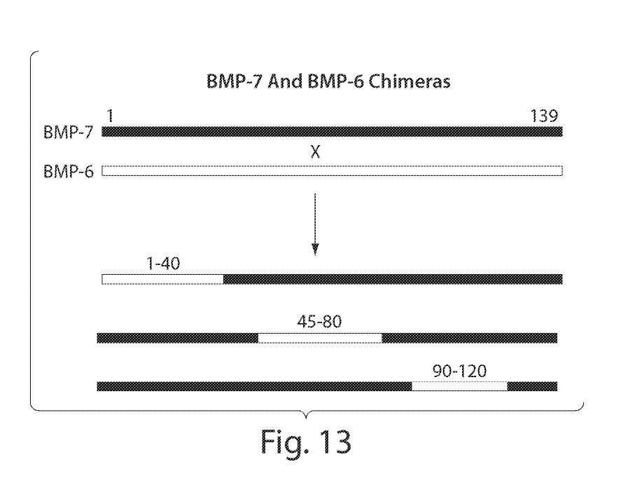

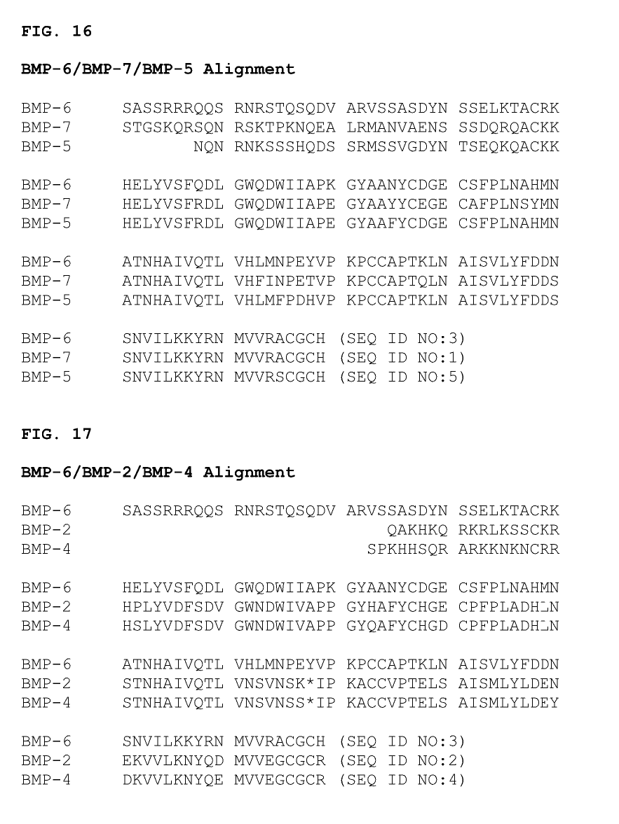

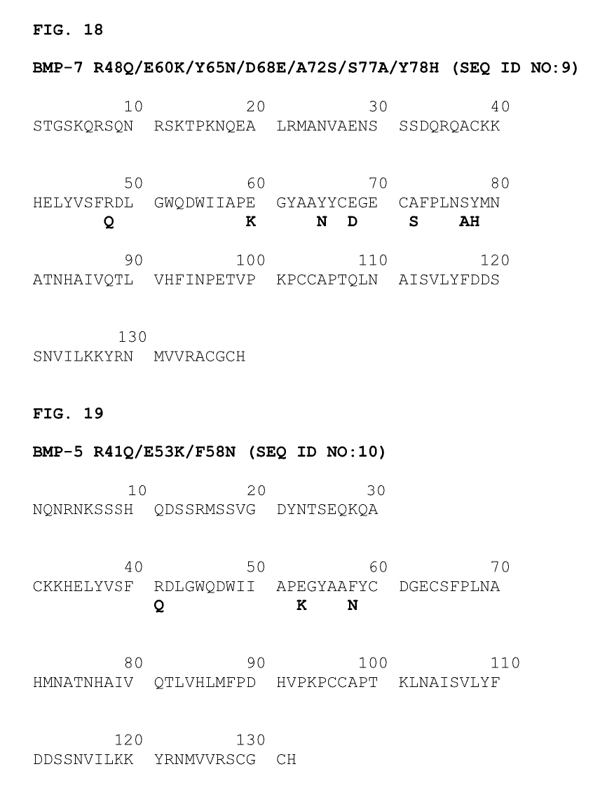

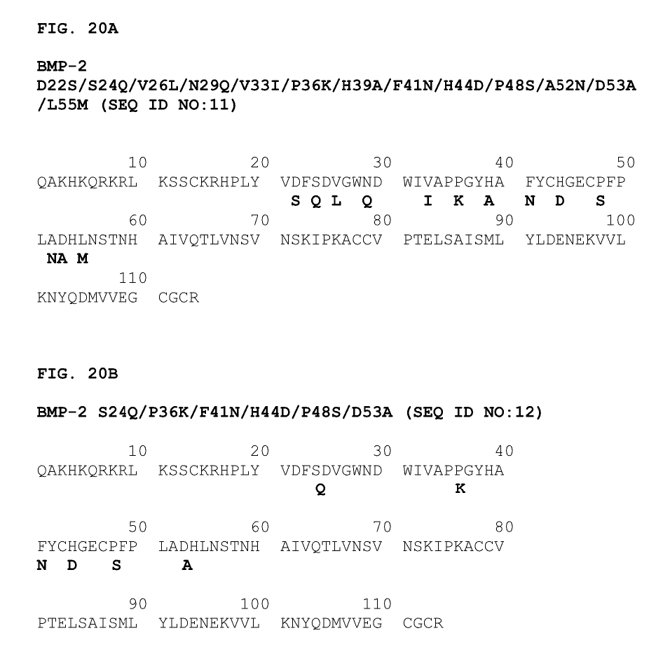

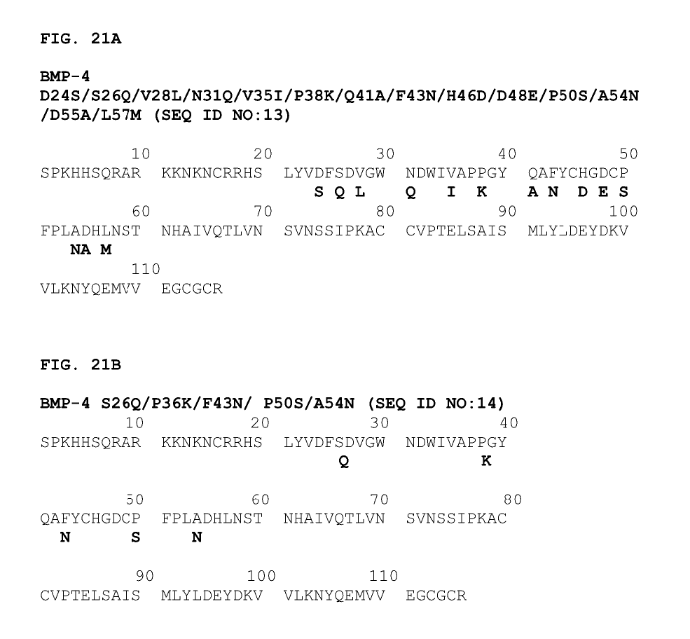

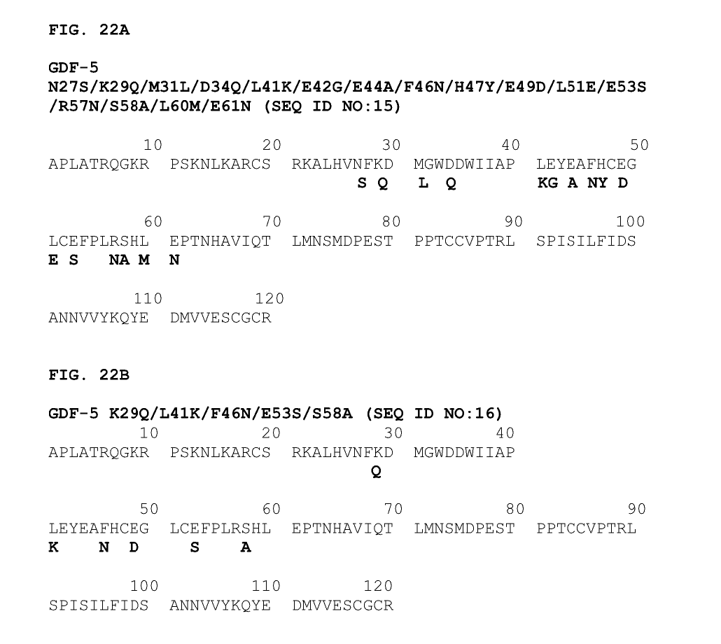

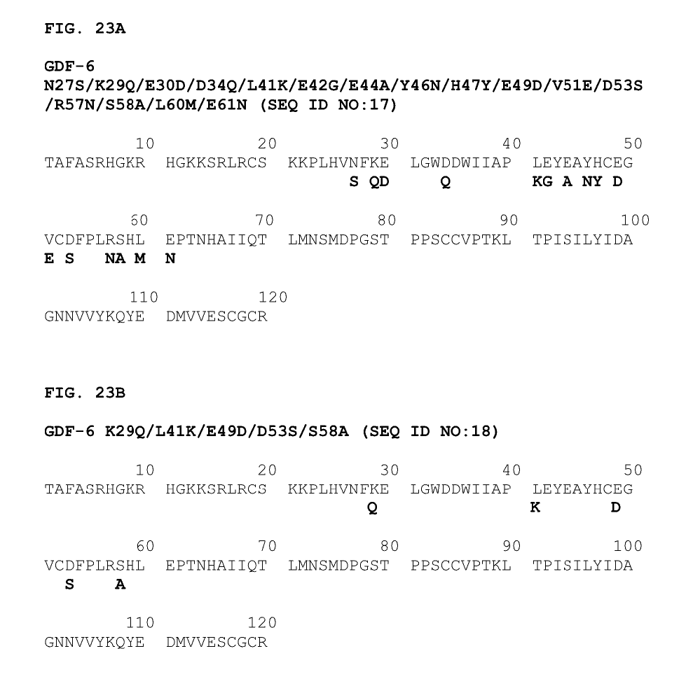

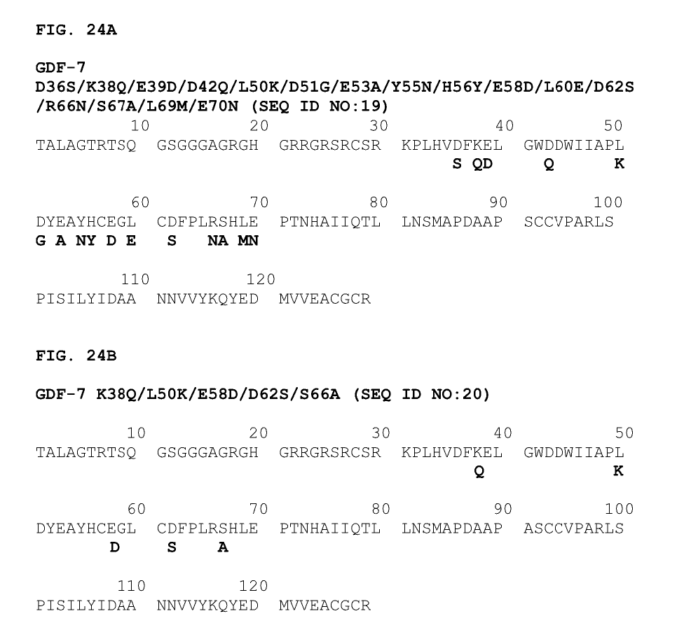

Given that BMP-6 is resistant to Noggin inhibition, determining the cause for this characteristic and harnessing it could permit engineering variants of other BMPs that are also resistant to Noggin. To this end, amino acid sequences of BMP-6 and its closest paralog, BMP-7, which is significantly more sensitive to noggin inhibition, were compared. FIG. 13 shows a schematic view of mature BMP-7 and BMP-6. These two proteins are highly homologous; however, divergent amino acids fall into the three regions depicted in FIG. 13: regions 1-40, 45-80 and 90-120. Sequence alignment of the BMP-6 and BMP-7 mature peptides revealed that these molecules differ the most at their amino terminus (residues 1-40 of FIG. 16). Beyond the first cysteine of the mature peptides, only 11 residues were found to be different between BMP-7 and BMP-6 (see FIG. 16).

Based on the alignment, three BMP-6/BMP-7 chimeras were engineered in order to identify the BMP-6 residues responsible for the increased resistance to noggin (see FIG. 13). The chimeric proteins harboring these differences were generated by recombinant reconstruction and domain swapping resulting in a BMP-6 fragment being placed in a BMP-7 sequence in place of its corresponding part in BMP-7. Specifically, three BMP-7 mutant proteins were created by substituting (1) the amino terminus region sequence Ser1-Lys40 of BMP-7 (residues 1-40 o SEQ ID NO:1) with the sequence Ser1-Lys40 of BMP6 (residues 1-40 of SEQ ID NO:3) (termed “40-1” chimera or variant) (29 amino acid differences); (2) the central amino acid region sequence Val45-Asn80 of BMP-7 (residues 45-80 of SEQ ID NO:1) with the sequence Val45-Asn80 of BMP-6 (residues 45-80 of SEQ ID NO:3) (termed the “80-1” chimera or variant) (7 amino acid differences); and (3) the carboxy-terminal amino acid region sequence Leu90-Ser120 of BMP-7 (residues 90-120 of SEQ ID NO:1) with the sequence Leu90-Asn120 of BMP-6 (residues 90-120 of SEQ ID NO:3) (termed the “90-1” chimera or variant) (5 amino acid differences) (see FIGS. 13 and 16).

The three chimeras were then expressed in HEK-293T cells by transient transfection. Conditioned media (CM) from transfected cells were collected and analyzed for recombinant protein expression subsequently tested for susceptibility to noggin inhibition. Western blot analysis, using a polyclonal antibody raised against the human BMP-7 mature peptide, detected properly processed chimeras in the conditioned media. Of these three mutant constructs, 40-1 and 80-1 were expressed at similar levels while 90-1 was expressed at a somewhat lover level in the HEK-293T cells.

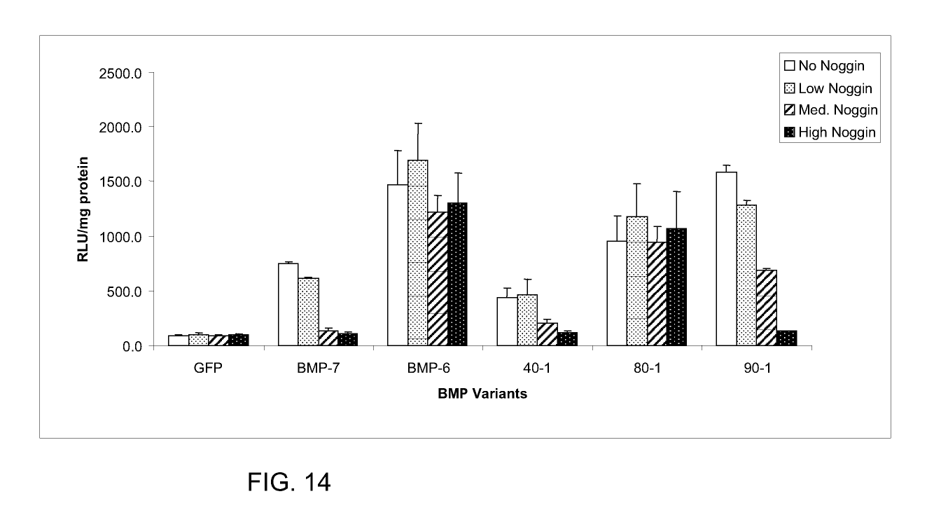

The conditioned media from transfected cells with plasmids expressing the three chimeras, as well as wild type BMP-6 and wild type BMP-7 were subsequently tested for their activity and sensitivity to noggin inhibition by the reporter gene assay in A549-BRE cells as previously described. Susceptibility to Noggin inhibition was assessed in the presence of three concentrations (N1=100 ng/ml, N2=500 ng/ml, and N3=1000 ng/ml) of noggin and the results are shown in FIG. 14.

As expected, BMP-7 was inhibited, in a dose-dependent manner while BMP-6 demonstrated marked resistance to noggin. Activity of 40-1 and 90-1 were inhibited by noggin by similar degrees to that of the wild type BMP-7. In contrast, and as observed for wild type BMP-6, the chimera 80-1 showed marked resistance to noggin inhibition at all three concentrations of Noggin. These results suggest that the BMP-6 sequence extending from residues 45 to 80 in the mature domain, which span part of finger 1 and wrist domains, is responsible for conferring noggin resistance. Moreover, it demonstrated that this specific characteristic of BMP-6 can be engineered into a different BMP by sequence manipulation.

To eliminate the possibility that the observed noggin resistance in the case of 80-1 is due to differences in expression levels or to the presence of other factors in the conditioned media, production of the 80-1 chimera was scaled up and the resulting protein was purified for further characterization. To this effect, a large scale transfection of 80-1 in HEK-293T cells was performed. The conditioned media was collected and the secreted 80-1 was purified using standard techniques.

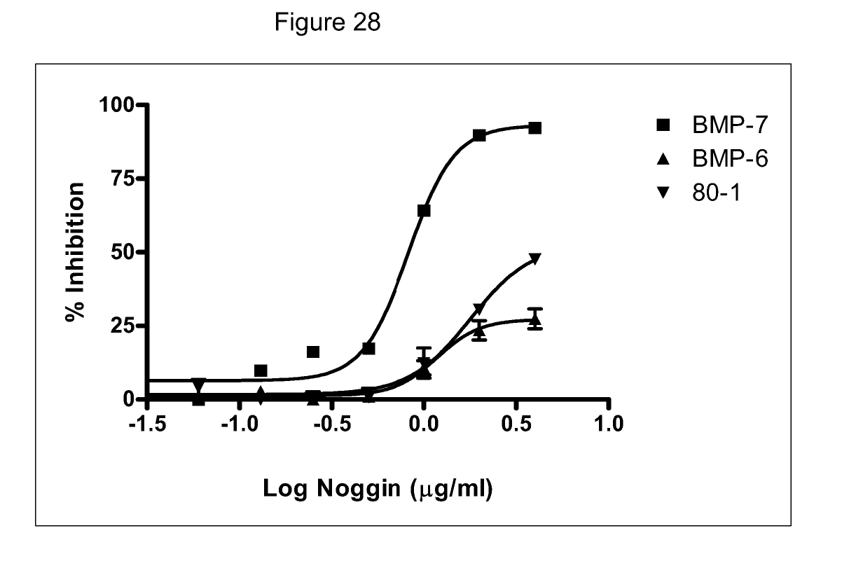

The activity and susceptibility to noggin of purified 80-1 was then compared side by side with purified wild-type BMP-6 and wild-type BMP-7. Each of the proteins (BMP-6, BMP-7, and 80-1) was tested at 100 ng/ml, in the presence of increasing concentrations of noggin. Full dose response curves for noggin were derived for each of the three proteins and are shown in FIG. 28. As expected, the purified 80-1 chimera was more resistant to Noggin inhibition than wild type BMP-7. The chimera's noggin response curve was similar to that of BMP-6, confirming the finding observed above using CM from transient transfections.

These results indicate that residues important for conferring resistance to Noggin inhibition are located in the stretch of amino acid sequence between residues Val45 and Asn80. As shown in the sequence alignments in FIG. 16, BMP-7 and BMP-6 differ in this region by seven amino acid residues. Thus 80-1 has the sequence of native BMP-7 with the following amino acid substitutions: R48Q, E60K, Y65N, E68D, A72S, S77A, and Y78H.

Further, given that of the three mutant constructs, 40-1 and 80-1 were expressed at similar levels while 90-1 was expressed at a somewhat lover level in the HEK-293T cells, mutating BMP-7 to have mutations found in the 80-1 mutant may provide increased expression of BMP-7 and may be useful for improving, even maximizing, production of BMP-7. For example, mutations at positions R48, E60, Y65, E68, A72, S77, and Y78 may contribute to increased expression of BMP-7 over the level of expression of wild-type BMP-7 in the same expression system. Also, double, trip, and quadruple mutants, i.e., mutants have modifications at two or more of positions R48, E60, Y65, E68, A72, S77, and Y78 may also confer increased levels of expression of mutant BMP-7s over wild-type BMP-7s in the same expression system. For example, a mutant BMP-7 having a modification at positions R48, E60, Y65 and A72 may confer an increased level of expression on the mutant BMP-7 over wild-type BMP-7 in the same expression system. Further, a mutant BMP-7 having a the modifications R48A, E60K, Y65N, and A72S may confer an increased level of expression on the mutant BMP-7 over wild-type BMP-7 in the same expression system.

### Example 5

### A Single Residue in BMP-6 Plays an Essential Role in Mediating Resistance to Noggin Inhibition

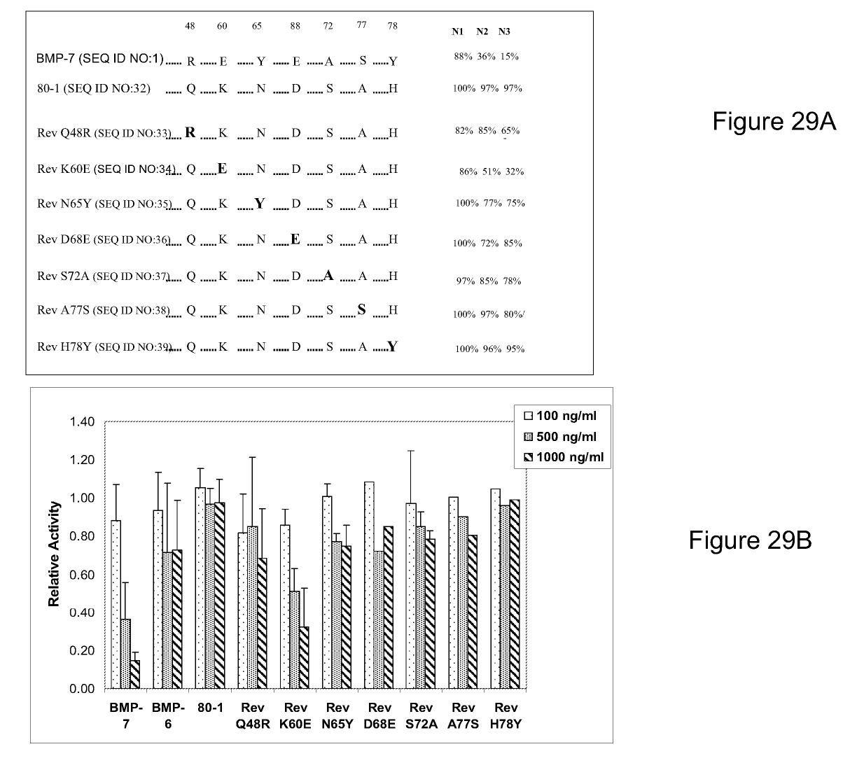

Alignment of BMP-6 and BMP-7 mature peptides showed that within the region extending from residues 45 to 80, seven residues are different between BMP-6 and BMP-7 (See FIG. 16). To further narrow down the residue(s) responsible for conferring noggin resistance, each of the seven residues in the 80-1 differing from wild-type BMP-7 was mutated back to its corresponding residue in BMP-7 as shown in FIG. 29A. Revertants of 80-1, (termed “Rev”), generated in this fashion were tested for their sensitivity to three concentrations of noggin (N1=100 ng/ml, N2=500 ng/ml, and N3=1000 ng/ml) as previously described above. The results are summarized in FIGS. 29A-B.

Five of the seven revertants of 80-1, Rev Q48R (SEQ ID NO:33), Rev D68E (SEQ ID NO:36), Rev S72A (SEQ ID NO:37), Rev A77S (SEQ ID NO:38), and Rev H78Y (SEQ ID NO:39) were as resistant to Noggin as 80-1 and wild-type BMP-6 as shown by the relative activity data presented for the three noggin dosages in FIGS. 29A and B. These data suggest that these residues are not essential for noggin resistance. In contrast, reverting lysine 60 back to a glutamic acid (Rev K60E) had a marked increase in susceptibility to noggin as compared to 80-1. As shown in FIGS. 29A-B, Rev K60E (SEQ ID NO:34) had relative activity of 86%, 51% and 32% as the concentration of Noggin increased. This results suggests that lysine 60 (K60) is a critical residue contributing to resistance to noggin inhibition.

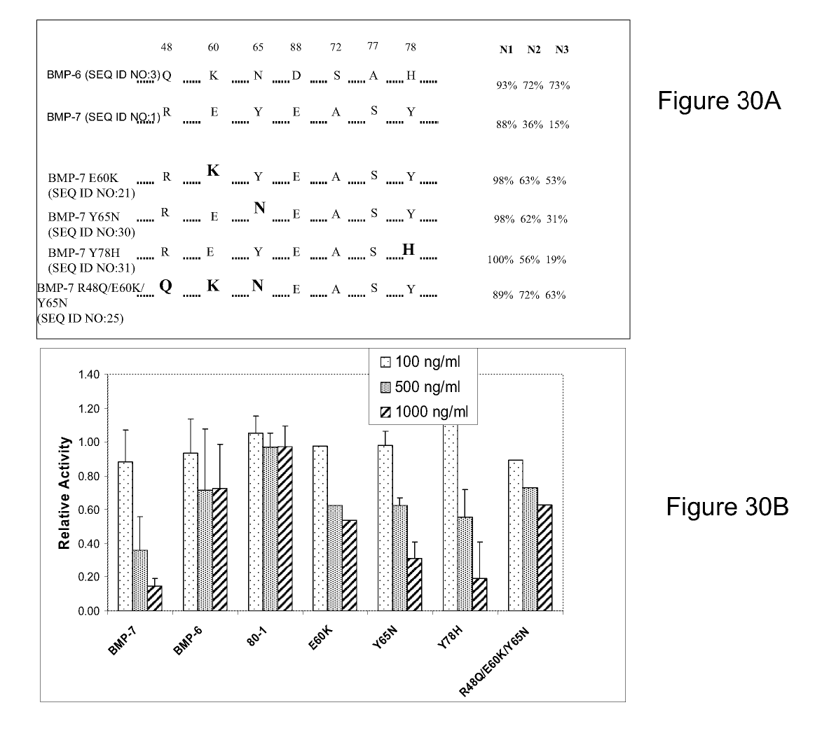

In another set of experiments, BMP-7 variants with single or multiple mutations targeting the residues differing between BMP-6 and BMP-7 within the region extending between residues 45 to 80 were created as shown in FIG. 30A. In these experiments, site directed mutagenesis was used to replace the BMP-7 residue(s) by their corresponding amino acids in the BMP-6 sequence. These mutants were subsequently expressed in 293T cells by transient transfection as previously described. Conditioned media containing these recombinant mutants were then screened for noggin resistance at three concentrations of noggin (N1=100 ng/ml, N2=500 ng/ml, and N3=1000 ng/ml) according to the previously described assay.

Consistent with the results obtained with the 80-1 revertants, the mutant BMP-7 E60K was more resistant to noggin than wild-type BMP-7. The mutant Y65N also had some, albeit minimal, effect on noggin resistance. For example, as shown in FIG. 30, while wild-type BMP-7 had only 15% relative activity at 1000 ng/ml of noggin, BMP-7 E60K had 53% activity. The mutant Y78H had no significant effect on sensitivity to noggin, providing relative activity of 31% at 1000 ng/ml of noggin as compared to 15% relative activity for wild-type BMP-7. These data further support that the lysine 60 (K60) plays a major role in conferring susceptibility to noggin inhibition.

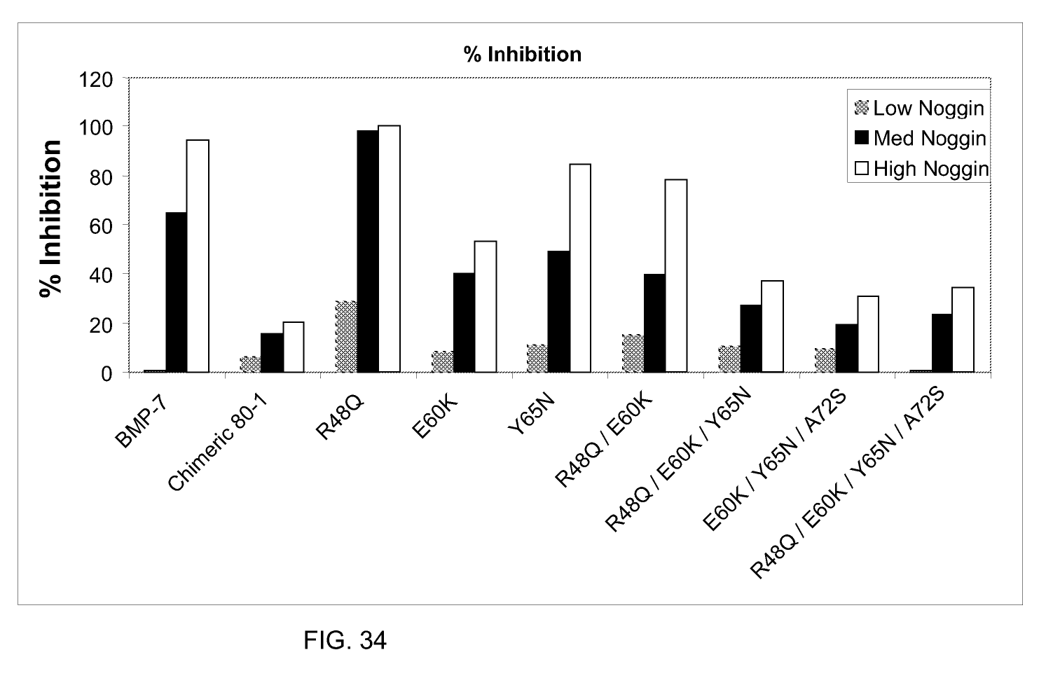

Interestingly, a triple mutant BMP-7 R48Q/E60K/Y65N (SEQ ID NO:25), which included mutations at positions 48, 60, and 65 further increased resistance to noggin. As shown in FIGS. 30A-B, relative activity of the triple mutant at 1000 ng/ml of noggin was 63%, quite close to the activity for BMP-6 at that concentration which was 73%. As shown in FIG. 34, while % inhibition by Noggin of BMP-7 mutants having only one each of the three point mutations demonstrated varying degrees of resistance to inhibition by noggin, the triple mutant showed % inhibition lower than the BMP-7 E60K mutant at the highest noggin concentration (FIG. 34). The data from triple mutant suggests that there may be synergy among these residues in mediating noggin sensitivity.

### Example 6

### Function of K60 in BMP-6 in Mediating Noggin Sensitivity is Conserved During Evolution

BMP interaction with extra-cellular antagonists is an important negative feed-back loop mechanism for regulating the activity of these growth factors. In order to determine if the BMP-7 glutamic acid residue at position 60—which was found to be critical for noggin susceptibility—is conserved among other BMPs, the amino acid sequences of a panel of BMPs including BMP-2, 4, 5, 6, 7, and 9 surrounding this residue were compared and are shown in FIG. 31A (amino acids numbered according to BMP-7). Only BMP-6 and BMP-9 sequences contain a lysine at position 60. In contrast, BMP-2, -4, -5, and -7 have either a proline residue or a glutamic acid residue in this position.

To confirm the importance of K60 in to conferring noggin resistance, we tested purified recombinant BMP-2, -6, -7, -9 and BMP-7 E60K for their susceptibility to noggin at three concentrations of noggin (N1=100 ng/ml, N2=500 ng/ml, and N3=1000 ng/ml). The results are shown in FIG. 31B. As demonstrated previously, BMP-2 was the most sensitive to noggin inhibition, followed by BMP-7. In contrast, BMP-6, BMP-9 and BMP-7 E60K were all resistant to noggin as demonstrated by their high relative activity in the presence of varying concentrations of noggin. These results further support the role of K60 in mediating noggin resistance.

Next, a lysine residue was introduced into the BMP-2 sequence at a position corresponding to the BMP-6 K60 to determine if this would generate a BMP-2 mutant with increased resistance to noggin. Such a mutant was generated and referred to as BMP-2/P36K.

The BMP-2 P36K mutant (SEQ ID NO:40) was transfected into 293T cells. Conditioned media from 293T cells transfected with either BMP-2/P36K and wild-type BMP-2 and wild-type BMP-6 constructs were then tested for noggin sensitivity at three concentrations of noggin (N1=100 ng/ml, N2=500 ng/ml, and N3=1000 ng/ml). The results are shown in FIG. 31C. As expected, BMP-2 P36K demonstrated a significant increase in noggin resistance compared to wild-type BMP-2. However, this BMP-2 mutant was still more sensitive to noggin than wild-type BMP-6, indicating that additional residues are needed for conferring the full noggin resistance in BMP-2.

Additional mutants with point mutations at residues corresponding to position 60 of BMP-6 are made and tested to determine if these mutations confer increased resistance to noggin inhibition. In particular, mutants BMP-4 P38K (SEQ ID NO:41), BMP-5 E53K (SEQ ID NO:42), GDF-5 L51K (SEQ ID NO:43), GDF-6 L51K (SEQ ID NO:44), and GDF-7 L50K (SEQ ID NO:45) are generated through standard techniques and transfected into 293T cells. Conditioned media from 293T cells transfected with these mutants, and along with wild-type BMP-6, BMP-4, BMP-5, GDF-5, GDF-6, and GDF-7 are tested for noggin sensitivity at three concentrations of noggin (N1=100 ng/ml, N2=500 ng/ml, and N3=1000 ng/ml). The results indicates that each of these mutants have increased resistance to noggin over their wild-type counterpart.

### Example 7

### Other Residues in BMP-6 Confer Noggin Resistance

In order to determine if residues in the portion of BMP-6 from residue 45-80 that differ with BMP-7 can induce noggin resistance in BMP-7 when combined together, various double, triple, and quadruple mutants were made. The BMP-7 mutant sequences are shown in FIG. 32.

Five mutants made included point mutations in addition to the E60K point mutation, allowing for a determination of other residues that contribute to noggin inhibition resistance. In particular, mutants BMP-7 R48Q/E60K (SEQ ID NO:23), BMP-7 R48Q/E60K/S77A (SEQ ID NO:24), BMP-7 R48Q/E60K/Y65N (SEQ ID NO: 25), BMP-7 E60K/Y65N (SEQ ID NO:26), BMP-7 E60K/Y65N/A72S (SEQ ID NO:27), and BMP-7 R48Q/E60K/Y65N/A72S (SEQ ID NO:28) were generated according to standard techniques and subsequently transfected into and expressed in 293T cells. Conditioned media from the transfected 293T cells containing the expressed mutants, and along with wild-type BMP-6 and BMP-7 were tested for sensitivity to noggin inhibition at three concentrations of noggin (N1=100 ng/ml, N2=500 ng/ml, and N3=1000 ng/ml). All of these five mutants conferred increased resistance to noggin inhibition as compared to wild-type BMP-7, (four of which are shown in FIG. 34), indicating that positions in addition to position 60 can confer noggin resistance. In particular the BMP-7 R48Q/E60K/Y65N/A72S (SEQ ID NO:28) exhibited the highest resistance to noggin inhibition compared to the other double and triple mutants as shown in FIG. 34.

Two mutants without mutations at position 60 were also made to determine if noggin resistance could be conferred without the presence of lysine at position 60 in BMP-7. In particular, the mutants BMP-7 R48Q/S77A (SEQ ID NO:22) and BMP-7 Y65N/Y78H (SEQ ID NO:29) were constructed according to standard techniques and subsequently transfected into and expressed in 293T cells. Conditioned media from the transfected cells containing the expressed mutants, and along with wild-type BMP-6 and BMP-7 were tested for sensitivity to noggin inhibition at three concentrations of noggin (N1=100 ng/ml, N2=500 ng/ml, and N3=1000 ng/ml). Each of these mutants conferred increased resistance to noggin inhibition as compared to wild-type BMP-7, indicating that other residues beyond the lysine at position 60 can contribute to Noggin resistance.

### Example 8

### BMP Mutants According to the Invention have In Vivo Activity

### Rat Model

The functioning of the various mutant BMPs of this invention are evaluated with an in vivo bioassay. The bioassay for bone induction as described by Sampath and Reddi ((1983) Proc. Natl. Acad. Sci. USA 80 6591 6595), herein incorporated by reference, is used to monitor endochondral bone differentiation activity of implanted mutants. This assay consists of implanting test samples in subcutaneous sites in recipient rats under ether anesthesia. Male Long-Evans rats, aged 28-32 days are used. A vertical incision (1 cm) is made under sterile conditions in the skin over the thoracic region, and a pocket is prepared by blunt dissection. Approximately 25 mg of the test sample of mutant BMP carried in a suitable matrix is implanted deep into the pocket and the incision is closed with a metallic skin clip. Control rats receive no implant. The day of implantation is designated as day one of the experiment. Implants are removed on day 12. The heterotropic site allows for the study of bone induction without the possible ambiguities resulting from the use of orthotropic sites.

Bone inducing activity is determined biochemically by the specific activity of alkaline phosphatase and calcium content of the day 12 implant. An increase in the specific activity of alkaline phosphatase indicates the onset of bone formation. Calcium content, on the other hand, is proportional to the amount of bone formed in the implant. Bone formation therefore is calculated by determining the calcium content of the implant on day 12 in rats and is expressed as “bone forming units,” where one bone forming unit represents the amount of protein that is needed for half maximal bone forming activity of the implant on day 12. Bone induction exhibited by intact demineralized rat bone matrix is considered to be the maximal bone differentiation activity for comparison purposes in this assay.

BMP mutants implanted exhibit a controlled progression through the stages of protein-induced endochondral bone development, including: (1) transient infiltration by polymorphonuclear leukocytes on day one; (2) mesenchymal cell migration and proliferation on days two and three; (3) chondrocyte appearance on days five and six; (4) cartilage matrix formation on day seven; (5) cartilage calcification on day eight; (6) vascular invasion, appearance of osteoblasts, and formation of new bone on days nine and ten; (7) appearance of osteoclasts, bone remodeling and dissolution of the implanted matrix on days twelve to eighteen; and (8) hematopoietic bone marrow differentiation in the ossicles on day twenty-one. The results show that the shape of the new bone conforms to the shape of the implanted matrix.

Histological sectioning and staining is preferred to determine the extent of osteogenesis in the implants. Implants are fixed in Bouins Solution, embedded in paraffin, and cut into 6-8 um sections. Staining with toluidine blue or hemotoxylin/eosin demonstrates clearly the ultimate development of endochondral bone in the twelve day implants of the mutant BMPs.

Alkaline phosphatase activity is used as a marker for osteogenesis. The enzyme activity is determined spectrophotometrically after homogenization of the implant. The activity peaks at 9-10 days in vivo and thereafter slowly declines. Implants showing no bone development by histology have little or no alkaline phosphatase activity under these assay conditions. The assay is useful for quantification and obtaining an estimate of bone formation quickly after the implants are removed from the rat. Alternatively, the amount of bone formation can be determined by measuring the calcium content of the implant.

Histological examination of implants containing mutant BMPs according to the invention indicate that these proteins have true osteogenic activity.

### Rabbit Model

Eight mature (less than 10 lbs) New Zealand White rabbits with epiphyseal closure documented by X-ray are studied. Defects of 1.5 cm are created in the rabbits, with implantation of matrices each containing one mutant BMP species according to the invention. Control defects have no implant placed.

Of the eight animals (one animal each was sacrificed at one and two weeks), 11 ulnae defects are followed for the full course of the eight week study. In all cases (n=7) following osteo-periosteal bone resection, the no implant animals establish no radiographic union by eight weeks. All no implant animals develop a thin “shell” of bone growing from surrounding bone present at four weeks and, to a slightly greater degree, by eight weeks. In all cases (n=4), radiographic union with marked bone induction is established in the BMP mutant-implanted animals by eight weeks. As opposed to the no implant repairs, this bone is in the site of the removed bone.

Radiomorphometric analysis reveals 90% BMP mutant-implant bone repair and 18% no-implant bone repair at sacrifice at eight weeks. At autopsy, the BMP mutant-implant bone appears normal, while “no implant” bone sites have only a soft fibrous tissue with no evidence of cartilage or bone repair in the defect site.

In another experiment, the marrow cavity of the 1.5 cm ulnar defect is packed with a BMP mutant protein of the invention and rabbit bone powder and the bones are allografted in an intercalary fashion. Control animals receive no protein and bone powder. The two negative control ulnae are not healed by eight weeks and reveal the classic “ivory” appearance. In distinct contrast, the BMP-treated implants “disappear” radiographically by four weeks with the start of remineralization by six to eight weeks. These allografts heal at each end with mild proliferative bone formation by eight weeks. Accordingly, this device with the mutant BMP according to the invention serves to accelerate allograph repair.

These studies of 1.5 cm osteo-periosteal defects in the ulnae of mature rabbits show that: (1) it is a suitable model for the study of bone growth; (2) “no implant” or negative control implants yield a small amount of periosteal-type bone, but not medullary or cortical bone growth; (3) BMP mutant-implanted rabbits exhibit proliferative bone growth in a fashion highly different from the control groups; (4) initial studies show that the bones exhibit 50% of normal bone strength (100% of normal correlated vol:vol) at only eight weeks after creation of the surgical defect; and (5) BMP mutant-allograft studies reveal a marked effect upon both the allograft and bone healing.

### Example 9

### BMP Mutants According to the Invention Induce Osteogenic Activity In Vivo in Lower Quantities than Wild-Type BMP

It has been observed that implant sites receiving BMP-7 (OP-1) have high levels of noggin as compared to non-implant sites. Accordingly, it is believed that the presence of high levels of BMPs, as experienced at an implant site, induce noggin expression. It is speculated BMP-7 is involved in a regulatory mechanism that increases noggin expression in order to neutralize the effect of BMP-7. Because high concentrations of BMP-7 are necessary to induce osteogenic and chondrogenic activity at implantation sites, it is predicted that a mutant BMP according to the invention that is not inhibited by noggin will be effective at inducing osteogenic and chondrogenic activity at an implant site in a lower concentration that it's wild-type counterpart. This is because BMPs will not be bound by noggin and prevented from activating the cascade of events that leads to bone and cartilage formation.

In order to verify this hypothesis, twelve mature (less than 10 lbs) New Zealand White rabbits are taken and defects of 1.5 cm are created in the ulnae. Four rabbits are implanted with a matrix containing 25 mg of BMP-7. Four rabbits are implanted with a matrix containing 25 mg of BMP-7 E60K. For the four control rabbits, the defects have no implant placed.

At two weeks, the rabbits are sacrificed and the ulnae are extracted. For control animals, a thin “shell” of bone growing from surrounding bone is present. Bone strength is tested and is found to be 5% of normal bone strength (100% of normal correlated vol:vol). For the BMP-7 matrix containing rabbit, while bone growth has occurred, the mean bone strength is 5% of normal bone strength. In contrast, for the BMP-7 mutant matrix rabbits, bone growth has occurred and mean bone strength is 50% of normal bone strength. Accordingly, given that equal concentrations of BMPs are provided on the implanted matrices, the mutant BMP-7 is able to induce more osteogenic growth in the same period of time which is represented by the increased bone strength of BMP-7 mutant implanted ulnae.

This result is verified by taking another approach. Twelve mature (less than 10 lbs) New Zealand White rabbits are taken and defects of 1.5 cm are created in the ulnae. Four rabbits are implanted with a matrix containing 100 mg of BMP-7. Four rabbits are implanted with a matrix containing 25 mg of BMP-7 E60K. For the four control rabbits, the defects have no implant placed.

At two weeks, the rabbits are sacrificed and the ulnae are extracted. For control animals, a thin “shell” of bone growing from surrounding bone is present. Mean bone strength is tested and is again found to be 5% of normal bone strength (100% of normal correlated vol:vol). For the BMP-7 matrix containing rabbit, bone growth has occurred and the mean bone strength is 45% of normal bone strength. For the BMP-7 mutant matrix rabbits, bone growth has occurred and mean bone strength is 50% of normal bone strength. Accordingly, the results show that a higher concentration of wild-type BMP-7 is necessary to induce the same level of osteogenic growth as a lower concentration of BMP-7 mutant of the invention over the same time period.

### Example 10

### BMP Mutants of the Invention are Effective at Inducing Bone and Cartilage Growth in Low Concentrations in Human Patients

Two human patients each require treatment to effect posterolateral fusion in the lumbar spine. In one patient, 1.5 mg of BMP-7 E60K in a matrix of bovine bone collagen and carboxymethylcellulose sodium (similar to OP-1® Putty, Stryker Biotech, Hopkinton, Mass.) is surgically implanted on each side of the spine at the site requiring fusion. The matrix is reconstituted with a sterile saline (0.9%) solution prior to implantation. In the other patient, 3.5 mg of wild-type BMP-7 in a matrix of bovine bone collagen and carboxymethylcellulose sodium (similar to OP-1®Putty, Stryker Biotech, Hopkinton, Mass.) is surgically implanted on each side of the spine at the site requiring fusion.

After a first period of several months, each patient's spine is viewed radiographically, for example, by X-ray to determine presence of bone growth at the fusion. In the patient receiving BMP-7 E60K, bone growth is detected at the fusion site. However, fusion is not complete. In the patient receiving wild-type BMP-7, the same level of bone growth is detected as in the patient receiving BMP-7 E60K. Again, fusion of the vertebrae is not complete.

After a second period of several months, equal to the first period of several months, each patient's spine is again viewed radiographically, for example, by X-ray. In each patient, fusion of the vertebrae at the site of implantation is complete.

Accordingly, mutant BMPs may be administered in lower concentrations than the corresponding wild-type BMPs while still promoting the same rate of bone growth. This may be attributed to the mutants' resistance to inhibition by noggin.

In another example, two human patients each require treatment to effect posterolateral fusion in the lumbar spine. In one patient, 3.5 mg of BMP-7 E60K in a matrix of bovine bone collagen and carboxymethylcellulose sodium (similar to OP-1®Putty, Stryker Biotech, Hopkinton, Mass.) is surgically implanted on each side of the spine at the site requiring fusion. The matrix is reconstituted with a sterile saline (0.9%) solution prior to implantation. In the other patient, 3.5 mg of wild-type BMP-7 in a matrix of bovine bone collagen and carboxymethylcellulose sodium (similar to OP-1®Putty, Stryker Biotech, Hopkinton, Mass.) is surgically implanted on each side of the spine at the site requiring fusion.

After a first period of several months, each patient's spine is viewed radiographically, for example, by X-ray to determine presence of bone growth at the fusion. In the patient receiving BMP-7 E60K, bone growth is detected at the fusion site and the fusion of the vertebrae is complete. In contrast, in the patient receiving wild-type BMP-7, bone growth is detected at the site of implantation. However, fusion of the vertebrae is not complete.

After a second period of several months, equal to the first period of several months, the patient receiving wild-type BMP-7's spine is again viewed radiographically, for example, by X-ray. Fusion of the vertebrae at the site of implantation is complete.

Accordingly, mutant BMPs may be administered in the same concentrations as the corresponding wild-type BMPs to achieve an accelerated rate of bone growth. This may be attributed to the mutants' resistance to inhibition by noggin.

### INCORPORATION BY REFERENCE

All sequence and structure access numbers, publications and patent documents cited in this application are incorporated by reference in their entirety for all purposes to the same extent as if the content of each individual publication or patent document was incorporated herein.

We claim:

1. A protein comprising a chimera of a TGF-beta superfamily protein and wild-type BMP-6, wherein one or more amino acid sequences of wild-type BMP-6 replace amino acid sequences at corresponding residue positions of the TGF-beta superfamily protein, wherein the chimera is resistant to inhibition by an antagonist of the TGF-beta superfamily protein and exhibits greater biological activity than that of the TGF-beta superfamily protein, and further wherein the TGF-beta superfamily protein is not wild-type BMP-6.

2. A protein comprising a modified bone morphogenetic protein (BMP) or a modified growth differentiation factor (GDF), wherein the modified BMP or GDF is less inhibited by an antagonist than a corresponding unmodified wild-type BMP or wild-type GDF and has greater biological activity than the corresponding unmodified wild-type BMP or GDF.

3. The protein of claim 1 or 2, wherein the antagonist is selected from the group of cysteine knot protein antagonists consisting of: Noggin, Noggin-like proteins, Cerberus/DAN family proteins, Chordin/SOG family proteins and functional equivalents of any of the foregoing.

4. A protein resistant to inhibition by Noggin or a Noggin-like protein, said protein comprising a modified bone morphogenetic protein (BMP) or a modified growth differentiation factor (GDF), wherein
(a) at least three replacement amino acids selected from the group consisting of Val45, Gln48, Asp49, Lys50, Gln53, Ile57, Lys60, Gly61, Ala63, Asn65, Tyr66, Asp68, Glu70, Ser72, Asn76, Ala77, His 78, Met79 and Asn80 of human BMP-6 replace at least three amino acids in corresponding positions of a wild-type BMP or a wild-type GDF, and wherein
(b) said at least three replacement amino acids differ from said at least three amino acids in corresponding positions of said wild-type BMP or said wild-type GDF.

5. A protein resistant to inhibition by Noggin or a Noggin-like protein, said protein comprising a modified bone morphogenetic protein (BMP) or a modified growth differentiation factor (GDF), wherein
(a) Val45 to Asn80 of wild-type human BMP-6 replaces a corresponding segment of an otherwise wild-type human BMP or human GDF and
(b) the corresponding segment that has been replaced is not identical to said Val45 to Asn80 of wild-type BMP-6.

6. A modified BMP-2 protein comprising at least one amino acid substitution selected from the group consisting of D22S, S24Q, V26L, N29Q, V33I, P36K, H39A, F41N, H44D, P48S, A52N, D53A, and L55M, wherein all other residues are identical to wild-type BMP-2 or share at least 93% amino acid sequence identity with the conserved cysteine domain of the C-terminal region of wild-type BMP-2.

7. A modified BMP-4 protein comprising at least one amino acid substitution selected from the group consisting of D24S, S26Q V28L, N31Q, V35I, P38K, Q41A, F43N, H46D, D48E, P50S, A54N, D55A, and L57M, wherein all other residues are identical to wild-type BMP-4 or share at least 93% amino acid sequence identity with the conserved cysteine domain of the C-terminal region of wild-type BMP-4.

8. A modified BMP-5 protein comprising at least one amino acid substitution selected from the group consisting of R41Q, E53K, and F58N, wherein all other residues are identical to wild-type BMP-5 or share at least 93% amino acid sequence identity with the conserved cysteine domain of the C-terminal region of wild-type BMP-5.

9. A modified BMP-7 protein comprising at least one amino acid substitution selected from the group consisting of R48Q, E60K, E68D, A72S and S77A, wherein all other residues are identical to wild-type BMP-7 or share at least 89% amino acid sequence identity with the conserved cysteine domain of the C-terminal region of wild-type BMP-7.

10. A modified BMP-7 protein comprising at least two amino acid substitutions selected from the group consisting of R48Q, E60K, Y65N, E68D, A72S and S77A, wherein all other residues are identical to wild-type BMP-7 or share at least 89% amino acid sequence identity with the conserved cysteine domain of the C-terminal region of wild-type BMP-7.

11. A modified BMP-7 protein comprising at least three amino acid substitutions selected from the group consisting of R48Q, E60K, Y65N, E68D, A72S, S77A, and Y78H, wherein all other residues are identical to wild-type BMP-7 or share at least 89% amino acid sequence identity with the conserved cysteine domain of C-terminal region of wild-type BMP-7.

12. A modified GDF-5 protein comprising at least one amino acid substitution selected from the group consisting of N27S, K29Q, M31L, D34Q, L41K, E42G, E44A, F46N, H47Y, E49D, L51E, E53S, R57N, S58A, L60M, and E61N, wherein all other residues are identical to wild-type GDF-5 or share at least 87% amino acid sequence identity with the conserved cysteine domain of the C-terminal region of wild-type GDF-5.

13. A modified GDF-6 protein comprising at least one amino acid substitution selected from the group consisting of N27S, K29Q, E30D, D34Q, L41K, E42G, E44A, Y46N, H47Y, E48D, V51E, D53S, R57N, S58A, L60M, and E61N, wherein all other residues are identical to wild-type GDF-6 or share at least 97% amino acid sequence identity with the conserved cysteine domain of the C-terminal region of wild-type GDF-6.

14. A modified GDF-7 protein comprising at least one amino acid substitution selected from the group consisting of D36S, K38Q, E39D, D43Q, L50K, D51G, E53A, Y55N, H56Y, E58D, L60E, D62S, R66N, S67A, L69M, E70N, wherein all other residues are identical to wild-type GDF-7 or share at least 87% amino acid sequence identity with the conserved cysteine domain of the C-terminal region of wild-type GDF-7.

15. A method for modulating inhibition of a modified bone morphogenetic protein (BMP) or a modified growth differentiation factor (GDF) by Noggin or a Noggin-like protein, the method comprising the step of:
providing a modified bone morphogenetic protein (BMP) or a modified growth differentiation factor (GDF), wherein
(a) at least two replacement amino acids selected from the group consisting of Val45, Gln48, Asp49, Lys50, Gln53, Ile57, Lys60, Gly61, Ala63, Asn65, Tyr66, Asp68, Glu70, Ser72, Asn76, Ala77, Met79 and Asn80 of human BMP-6 replace at least two amino acids in corresponding positions of a wild-type BMP or a wild-type GDF, and
(b) said at least two replacement amino acids differ from said at least two amino acids in corresponding positions of said wild-type BMP or said wild-type GDF, and wherein
(c) the providing step results in a modulation of inhibition of said BMP or GDF by Noggin or a Noggin-like protein as compared to the BMP or GDF wild-type counterpart.

16. A method for modulating the inhibition of a bone morphogenetic protein (BMP) or a growth differentiation factor (GDF) by Noggin or a Noggin-like protein, the method comprising the step of:
providing a modified BMP or GDF protein, said modified protein resulting from replacing a corresponding segment of a human BMP or a human GDF with Val45 to Asn80 of wild-type human BMP-6, wherein the corresponding segment that has been replaced is not identical to said Val45 to Asn80 of wild-type BMP-6, wherein the providing step results in a modulation of inhibition of said BMP or GDF by Noggin or a Noggin-like protein as compared to the BMP or GDF wild-type counterpart.

17. A non-naturally occurring peptide comprising an N-terminal and a C-terminal amino acid sequence corresponding to a wild-type BMP or a GDF protein, wherein
(a) each of the N-terminal and the C-terminal amino acid sequences of said non-naturally occurring peptide shares at least 97% amino acid sequence identity with the wild-type BMP or GDF protein and further wherein
(b) said non-naturally occurring peptide has at least two amino acid residues at positions corresponding to BMP-6 amino acid residues selected from the group consisting of Val45, Gln48, Asp49, Lys50, Gln53, Ile57, Lys60, Gly61, Ala63, Asn65, Tyr66, Asp68, Glu70, Ser72, Asn76, Ala77, Met79 and Asn80, with the proviso that said BMP is not BMP-6.

18. The non-naturally occurring peptide of claim 17, wherein said N-terminal or said C-terminal amino acid sequence is identical to the wild-type BMP or GDF protein.

19. The non-naturally occurring peptide of claim 17, wherein said peptide displays reduced inhibition of bioactivity by a Noggin or Noggin-like protein as compared to its wild-type BMP or GDF counterpart.

20. A pharmaceutical composition comprising the peptide of claim 17 admixed with a pharmaceutically-acceptable carrier.

21. The modified BMP-2 of claim 6, comprising the amino acid substitution P36K.

22. The modified BMP-4 of claim 7, comprising the amino acid substitution P38K.

23. The modified BMP-5 of claim 8, comprising the amino acid substitution E53K.

24. The modified BMP-7 of claim 9, comprising the amino acid substitution E60K.

25. The modified BMP-7 of claim 10, comprising the amino acid substitutions R48Q and S77A.

26. The modified BMP-7 of claim 10, comprising the amino acid substitutions R48Q and E60K.

27. The modified BMP-7 of claim 10, comprising the amino acid substitutions E60K and Y765N.

28. The modified BMP-7 of claim 10, comprising the amino acid substitutions Y65N and Y78H.

29. The modified BMP-7 of claim 28, further comprising the amino acid substitution R134E.

30. The modified BMP-7 of claim 11, comprising the amino acid substitutions R48Q, E60K, and S77A.

31. The modified BMP-7 of claim 11, comprising the amino acid substitutions R48Q, E60K, and Y65N.

32. The modified BMP-7 of claim 11, comprising the amino acid substitutions E60K, Y65N, and A72S.

33. The modified BMP-7 of claim 11, comprising the amino acid substitutions R48Q, E60K, Y65N, and A72S.

34. The modified GDF-5 of claim 12, comprising the amino acid substitution L41K.

35. The modified GDF-6 of claim 13, comprising the amino acid substitution L41K.

36. The modified GDF-7 of claim 14, comprising the amino acid substitution L50K.

37. A nucleic acid encoding any one of the proteins of claims 1-14 and 21-36.

38. A vector comprising the nucleic acid of claim 37.

39. A pharmaceutical composition comprising any one of the proteins of claims 1-14 and 21-36 admixed with a pharmaceutically acceptable carrier.

40. A method of treating a patient in need of bone or cartilage repair comprising administering to the patient the pharmaceutical composition of claim 39.

41. The method of claim 40, wherein the patient is a human.

42. A modified BMP-7 comprising an amino acid substitution at least one position selected from the group consisting of R48, E60, Y65, E68, A72, S77, and Y78, wherein said modified BMP-7 has BMP-7 activity.

43. The modified BMP-7 of claim 42, wherein a substitution occurs at position 60.

44. The modified BMP-7 of claim 43, wherein the substitution at position 60 is a K, R, H, N, or Q residue.

45. The modified BMP-7 of claim 42, wherein a substitution occurs at position 48.

46. The modified BMP-7 of claim 45, wherein the substitution at position 48 is a Q, N, W, Y, or F residue.

47. The modified BMP-7 of claim 42, wherein a substitution occurs at position 65.

48. The modified BMP-7 of claim 47, wherein the substitution at position 65 is an N, Q, H, K, or R residue.

49. The modified BMP-7 of claim 42, wherein a substitution occurs at position 72.

50. The modified BMP-7 of claim 49, wherein the substitution at position 72 is an S, T, Y, N, or Q residue.

51. The modified BMP-7 of claim 42, wherein a substitution occurs at positions 48, 60, 65, and 72.

52. The modified BMP-7 of claim 42, wherein a substitution occurs at positions 48, 60, and 65.

53. A method for modulating inhibition of a modified bone morphogenetic protein (BMP) or a modified growth differentiation factor (GDF) by Noggin or a Noggin-like protein, the method comprising the step of:
providing a modified bone morphogenetic protein (BMP) or a modified growth differentiation factor (GDF),
(a) wherein at least two replacement amino acids selected from the group consisting of Asn77, Glu79, Ile81, Asp84, Ser85, Ile88, Lys91, Glu92, Glu94, Tyr96, Glu97, Lys99, Gly101, Phe103, Ala107, Asp108, Asp109, Val110, or Thr111 of human BMP-9 replace at least two amino acids in corresponding positions of a wild-type BMP or a wild-type GDF, and
(b) said at least two replacement amino acids differ from said at least two amino acids in corresponding positions of said wild-type BMP or said wild-type GDF, and also wherein
(c) the providing step results in a modulation of inhibition of said BMP or GDF by Noggin or a Noggin-like protein as compared to the BMP or GDF wild-type counterpart.

54. A method for modulating the inhibition of a bone morphogenetic protein (BMP) or a growth differentiation factor (GDF) by Noggin or a Noggin-like protein, the method comprising the step of:
providing a modified BMP or GDF protein, said modified protein resulting from replacing a corresponding segment of a human BMP or a human GDF with Val76 to Thr111 of wild-type human BMP-9, wherein the corresponding segment that has been replaced is not identical to said Val76 to Thr111 of wild-type BMP-9, and wherein the providing step results in a modulation of inhibition of said BMP or GDF by Noggin or a Noggin-like protein as compared to the BMP or GDF wild-type counterpart.

55. A non-naturally occurring peptide comprising an N-terminal and a C-terminal amino acid sequence corresponding to a wild-type BMP or a GDF protein, wherein
(a) each of the N-terminal and the C-terminal amino acid sequences of said non-naturally occurring peptide shares at least 97% amino acid sequence identity with the wild-type BMP or GDF protein and further wherein
(b) said non-naturally occurring peptide has at least two amino acid residues at positions corresponding to BMP-9 amino acid residues selected from the group consisting of Asn77, Glu79, Ile81, Asp84, Ser85, Ile88, Lys91, Glu92, Glu94, Tyr96, Glu97, Lys99, Gly101, Phe103, Ala107, Asp108, Asp109, Val110, or Thr111 and wherein said BMP is not BMP-9.


SEQUENCE LISTING (46 sequences)

SEQ ID NO: 1 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - protein, 139 aa, Homo sapiens
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 4 - protein, 116 aa, Homo sapiens
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 5 - protein, 132 aa, Homo sapiens
NQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 6 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 7 - protein, 120 aa, Homo sapiens
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 8 - protein, 129 aa, Homo sapiens
TALAGTRTSQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 9 - protein, 139 aa, Artificial Sequence
  Modified BMP-7 sequence
STGSKQRSQNRSKTPKNQEALRMANVAENSSSDQRQACKKHELYVSFQDLGWQDWIIAPKGYAANYCDGECSFPLNAHMNATNHAIVQTLVHFINPETVPKPCCAPTQLNAISVLYFDDSSNVILKKYRNMVVRACGCH

SEQ ID NO: 10 - protein, 132 aa, Artificial Sequence
  Modified BMP-5 sequence
NQNRNKSSSHQDSSRMSSVGDYNTSEQKQACKKHELYVSFQDLGWQDWIIAPKGYAANYCDGECSFPLNAHMNATNHAIVQTLVHLMFPDHVPKPCCAPTKLNAISVLYFDDSSNVILKKYRNMVVRSCGCH

SEQ ID NO: 11 - protein, 114 aa, Artificial Sequence
  Modified BMP-2 sequence
QAKHKQRKRLKSSCKRHPLYVSFQDLGWQDWIIAPKGYAANYCDGECSFPLNAHMNSTNHAIVQTLVNSVNSKIPKACCVPTELSAISMLYLDENEKVVLKNYQDMVVEGCGCR

SEQ ID NO: 12 - protein, 114 aa, Artificial Sequence
  Modified BMP-2 sequence
QAKHKQRKRLKSSCKRHPLYVDFQDVGWNKWIVAPKGYHANYCDGECSFPLAAHLNSTNHAIVQTLVNSVNSKIPKACCVPTELSAISMLYLDENEKVVLKNYQDMVVEGCGCR

SEQ ID NO: 13 - protein, 116 aa, Artificial Sequence
  Modified BMP-4 sequence
SPKHHSQRARKKNKNCRRHSLYVSFQDLGWQDWIIAPKGYAANYCDGECSFPLNAHMNSTNHAIVQTLVNSVNSSIPKACCVPTELSAISMLYLDEYDKVVLKNYQEMVVEGCGCR

SEQ ID NO: 14 - protein, 116 aa, Artificial Sequence
  Modified BMP-4 sequence
SPKHHSQRARKKNKNCRRHSLYVDFQDVGWNDWIVAPKGYQANYCHGDCSFPLNDHLNSTNHAIVQTLVNSVNSSIPKACCVPTELSAISMLYLDEYDKVVLKNYQEMVVEGCGCR

SEQ ID NO: 15 - protein, 120 aa, Artificial Sequence
  Modified GDF-5 sequence
APLATRQGKRPSKNLKARCSRKALHVSFQDLGWQDWIIAPKGYAANYCDGECSFPLNAHMNPTNHAVIQTLMNSMDPESTPPTCCVPTRLSPISILFIDSANNVVYKQYEDMVVESCGCR

SEQ ID NO: 16 - protein, 120 aa, Artificial Sequence
  Modified GDF-5 sequence
APLATRQGKRPSKNLKARCSRKALHVNFQDMGWDDWIIAPKEYEANHCDGLCSFPLRAHLEPTNHAVIQTLMNSMDPESTPPTCCVPTRLSPISILFIDSANNVVYKQYEDMVVESCGCR

SEQ ID NO: 17 - protein, 120 aa, Artificial Sequence
  Modified GDF-6 sequence
TAFASRHGKRHGKKSRLRCSKKPLHVSFQDLGWQDWIIAPKGYAANYCDGECSFPLNAHMNPTNHAIIQTLMNSMDPGSTPPSCCVPTKLTPISILYIDAGNNVVYKQYEDMVVESCGCR

SEQ ID NO: 18 - protein, 120 aa, Artificial Sequence
  Modified GDF-6 sequence
TAFASRHGKRHGKKSRLRCSKKPLHVNFQELGWDDWIIAPKEYEAYHCDGVCSFPLRAHLEPTNHAIIQTLMNSMDPGSTPPSCCVPTKLTPISILYIDAGNNVVYKQYEDMVVESCGCR

SEQ ID NO: 19 - protein, 128 aa, Artificial Sequence
  Modified GDF-7 sequence
TALAGTRTSQGSGGGAGRGHGRRGRSRCSRKPLHVSFQDLGWQDWIIAPKGYAANYCDGECSFPLNAHMNPTNHAIIQTLLNSMAPDAAPSCCVPARLSPISILYIDAANNVVYKQYEDMVVEACGCR

SEQ ID NO: 20 - protein, 129 aa, Artificial Sequence
  Modified GDF-7 sequence
TALAGTRTSQGSGGGAGRGHGRRGRSRCSRKPLHVDFQELGWDDWIIAPKDYEAYHCDGLCSFPLRAHLEPTNHAIIQTLLNSMAPDAAPASCCVPARLSPISILYIDAANNVVYKQYEDMVVEACGCR

SEQ ID NO: 21 - protein, 139 aa, Artificial Sequence
  BMP-7 E60K
STGSKQRSQNRSKTPKNQEALRMANVAENSSSDQRQACKKHELYVSFRDLGWQDWIIAPKGYAAYYCEGECAFPLNSYMNATNHAIVQTLVHFINPETVPKPCCAPTQLNAISVLYFDDSSNVILKKYRNMVVRACGCH

SEQ ID NO: 22 - protein, 139 aa, Artificial Sequence
  BMP-7 R48Q/S77A
STGSKQRSQNRSKTPKNQEALRMANVAENSSSDQRQACKKHELYVSFQDLGWQDWIIAPEGYAAYYCEGECAFPLNAYMNATNHAIVQTLVHFINPETVPKPCCAPTQLNAISVLYFDDSSNVILKKYRNMVVRACGCH

SEQ ID NO: 23 - protein, 139 aa, Artificial Sequence
  BMP-7 R48Q/E60K
STGSKQRSQNRSKTPKNQEALRMANVAENSSSDQRQACKKHELYVSFQDLGWQDWIIAPKGYAAYYCEGECAFPLNSYMNATNHAIVQTLVHFINPETVPKPCCAPTQLNAISVLYFDDSSNVILKKYRNMVVRACGCH

SEQ ID NO: 24 - protein, 139 aa, Artificial Sequence
  BMP-7 R48Q/E60K/S77A
STGSKQRSQNRSKTPKNQEALRMANVAENSSSDQRQACKKHELYVSFQDLGWQDWIIAPKGYAAYYCEGECAFPLNAYMNATNHAIVQTLVHFINPETVPKPCCAPTQLNAISVLYFDDSSNVILKKYRNMVVRACGCH

SEQ ID NO: 25 - protein, 139 aa, Artificial Sequence
  BMP-7 R48Q/E60K/Y65N
STGSKQRSQNRSKTPKNQEALRMANVAENSSSDQRQACKKHELYVSFQDLGWQDWIIAPKGYAANYCEGECAFPLNSYMNATNHAIVQTLVHFINPETVPKPCCAPTQLNAISVLYFDDSSNVILKKYRNMVVRACGCH

SEQ ID NO: 26 - protein, 139 aa, Artificial Sequence
  BMP-7 E60K/Y65N
STGSKQRSQNRSKTPKNQEALRMANVAENSSSDQRQACKKHELYVSFRDLGWQDWIIAPKGYAANYCEGECAFPLNSYMNATNHAIVQTLVHFINPETVPKPCCAPTQLNAISVLYFDDSSNVILKKYRNMVVRACGCH

SEQ ID NO: 27 - protein, 139 aa, Artificial Sequence
  BMP-7 E60K/Y65N/A72S
STGSKQRSQNRSKTPKNQEALRMANVAENSSSDQRQACKKHELYVSFRDLGWQDWIIAPKGYAANYCEGECSFPLNSYMNATNHAIVQTLVHFINPETVPKPCCAPTQLNAISVLYFDDSSNVILKKYRNMVVRACGCH

SEQ ID NO: 28 - protein, 139 aa, Artificial Sequence
  BMP-7 R48Q/E60K/Y65N/A72S
STGSKQRSQNRSKTPKNQEALRMANVAENSSSDQRQACKKHELYVSFQDLGWQDWIIAPKGYAANYCEGECSFPLNSYMNATNHAIVQTLVHFINPETVPKPCCAPTQLNAISVLYFDDSSNVILKKYRNMVVRACGCH

SEQ ID NO: 29 - protein, 139 aa, Artificial Sequence
  BMP-7 Y65N/Y78H
STGSKQRSQNRSKTPKNQEALRMANVAENSSSDQRQACKKHELYVSFRDLGWQDWIIAPEGYAANYCEGECAFPLNSHMNATNHAIVQTLVHFINPETVPKPCCAPTQLNAISVLYFDDSSNVILKKYRNMVVRACGCH

SEQ ID NO: 30 - protein, 139 aa, Artificial Sequence
  BMP-7 Y65N
STGSKQRSQNRSKTPKNQEALRMANVAENSSSDQRQACKKHELYVSFRDLGWQDWIIAPEGYAANYCEGECAFPLNSYMNATNHAIVQTLVHFINPETVPKPCCAPTQLNAISVLYFDDSSNVILKKYRNMVVRACGCH

SEQ ID NO: 31 - protein, 139 aa, Artificial Sequence
  BMP-7 Y78H
STGSKQRSQNRSKTPKNQEALRMANVAENSSSDQRQACKKHELYVSFRDLGWQDWIIAPEGYAAYYCEGECAFPLNSHMNATNHAIVQTLVHFINPETVPKPCCAPTQLNAISVLYFDDSSNVILKKYRNMVVRACGCH

SEQ ID NO: 32 - protein, 139 aa, Artificial Sequence; identical to SEQ ID NO: 9
  BMP-7 80-1

SEQ ID NO: 33 - protein, 139 aa, Artificial Sequence
  BMP-7 80-1 Rev Q48R
STGSKQRSQNRSKTPKNQEALRMANVAENSSSDQRQACKKHELYVSFRDLGWQDWIIAPKGYAANYCDGECSFPLNAHMNATNHAIVQTLVHFINPETVPKPCCAPTQLNAISVLYFDDSSNVILKKYRNMVVRACGCH

SEQ ID NO: 34 - protein, 139 aa, Artificial Sequence
  BMP-7 80-1 Rev K60E
STGSKQRSQNRSKTPKNQEALRMANVAENSSSDQRQACKKHELYVSFQDLGWQDWIIAPEGYAANYCDGECSFPLNAHMNATNHAIVQTLVHFINPETVPKPCCAPTQLNAISVLYFDDSSNVILKKYRNMVVRACGCH

SEQ ID NO: 35 - protein, 139 aa, Artificial Sequence
  BMP-7 80-1 Rev N65Y
STGSKQRSQNRSKTPKNQEALRMANVAENSSSDQRQACKKHELYVSFQDLGWQDWIIAPKGYAAYYCDGECSFPLNAHMNATNHAIVQTLVHFINPETVPKPCCAPTQLNAISVLYFDDSSNVILKKYRNMVVRACGCH

SEQ ID NO: 36 - protein, 139 aa, Artificial Sequence
  BMP-7 80-1 D68E
STGSKQRSQNRSKTPKNQEALRMANVAENSSSDQRQACKKHELYVSFQDLGWQDWIIAPKGYAANYCEGECSFPLNAHMNATNHAIVQTLVHFINPETVPKPCCAPTQLNAISVLYFDDSSNVILKKYRNMVVRACGCH

SEQ ID NO: 37 - protein, 139 aa, Artificial Sequence
  BMP-7 80-1 S72A
STGSKQRSQNRSKTPKNQEALRMANVAENSSSDQRQACKKHELYVSFQDLGWQDWIIAPKGYAANYCDGECAFPLNAHMNATNHAIVQTLVHFINPETVPKPCCAPTQLNAISVLYFDDSSNVILKKYRNMVVRACGCH

SEQ ID NO: 38 - protein, 139 aa, Artificial Sequence
  BMP-7 80-1 A77S
STGSKQRSQNRSKTPKNQEALRMANVAENSSSDQRQACKKHELYVSFQDLGWQDWIIAPKGYAANYCDGECSFPLNSHMNATNHAIVQTLVHFINPETVPKPCCAPTQLNAISVLYFDDSSNVILKKYRNMVVRACGCH

SEQ ID NO: 39 - protein, 139 aa, Artificial Sequence
  BMP-7 80-1 H78Y
STGSKQRSQNRSKTPKNQEALRMANVAENSSSDQRQACKKHELYVSFQDLGWQDWIIAPKGYAANYCDGECSFPLNAYMNATNHAIVQTLVHFINPETVPKPCCAPTQLNAISVLYFDDSSNVILKKYRNMVVRACGCH

SEQ ID NO: 40 - protein, 114 aa, Artificial Sequence
  BMP-2 P36K
QAKHKQRKRLKSSCKRHPLYVDFSDVGWNDWIVAPKGYHAFYCHGECPFPLADHLNSTNHAIVQTLVNSVNSKIPKACCVPTELSAISMLYLDENEKVVLKNYQDMVVEGCGCR

SEQ ID NO: 41 - protein, 116 aa, Artificial Sequence
  BMP-4 P38K
SPKHHSQRARKKNKNCRRHSLYVDFSDVGWNDWIVAPKGYQAFYCHGDCPFPLADHLNSTNHAIVQTLVNSVNSSIPKACCVPTELSAISMLYLDEYDKVVLKNYQEMVVEGCGCR

SEQ ID NO: 42 - protein, 132 aa, Artificial Sequence
  BMP-5 E53K
NQNRNKSSSHQDSSRMSSVGDYNTSEQKQACKKHELYVSFRDLGWQDWIIAPKGYAAFYCDGECSFPLNAHMNATNHAIVQTLVHLMFPDHVPKPCCAPTKLNAISVLYFDDSSNVILKKYRNMVVRSCGCH

SEQ ID NO: 43 - protein, 120 aa, Artificial Sequence
  GDF-5 L41K
APLATRQGKRPSKNLKARCSRKALHVNFKDMGWDDWIIAPKEYEAFHCEGLCEFPLRSHLEPTNHAVIQTLMNSMDPESTPPTCCVPTRLSPISILFIDSANNVVYKQYEDMVVESCGCR

SEQ ID NO: 44 - protein, 120 aa, Artificial Sequence
  GDF-6 L41K
TAFASRHGKRHGKKSRLRCSKKPLHVNFKELGWDDWIIAPLEKEAYHCEGVCDFPLRSHLEPTNHAIIQTLMNSMDPGSTPPSCCVPTKLTPISILYIDAGNNVVYKQYEDMVVESCGCR

SEQ ID NO: 45 - protein, 129 aa, Artificial Sequence
  GDF-7 L50K
TALAGTRTSQGSGGGAGRGHGRRGRSRCSRKPLHVDFKELGWDDWIIAPKDYEAYHCEGLCDFPLRSHLEPTNHAIIQTLLNSMAPDAAPASCCVPARLSPISILYIDAANNVVYKQYEDMVVEACGCR

SEQ ID NO: 46 - protein, 171 aa, Homo sapiens
NDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR